(19) United States
(12) Patent Application Publication
(10) Pub. No.: US 2004/0105858 A1
(43) Pub. Date: Jun. 3, 2004

(54) Diagnosis and treatment of infertility

(76) Inventors: Joanne Young Hee Kwak Kim, Lincolnshire, IL (US); Alan E. Beer, Lake Forest, IL (US); Alice Gilman-Sachs, Chicago, IL (US)

Correspondence Address: Bell, Boyd & Lloyd LLC, P.O. Box 1135, Chicago, IL, 60690-1135, US

(21) Appl. No.: 10/651,690

(22) Filed: Aug. 28, 2003

Related U.S. Application Data
(60) Provisional application No. 60/406,804, filed on Aug. 29, 2002.

(51) Int. Cl. A61K 39/395

(52) U.S. Cl. 424/144.1

(57) ABSTRACT

The present invention relates generally to the regulation of immune responses of subjects for the diagnosis and treatment of infertility. More particularly, methods that downregulate T helper 1 (Th1) immunity or upregulate T helper 2 (Th2) immunity are used to enhance reproductive outcomes in subjects with recurrent spontaneous abortions or implantation failures by changing the balance of T helper 1 and T helper 2 immune responses. Ratios of Th1 and Th2 activities can also be used for diagnosis of infertility in these subjects.

### CROSS-REFERENCE TO RELATED APPLICATIONS

This application claims priority from provisional application serial No. 60/406,804 filed Aug. 29, 2002, which is incorporated herein by reference and made a part hereof.

### FEDERALLY SPONSORED RESEARCH OR DEVELOPMENT

Not Applicable.

### FIELD OF THE INVENTION

The present invention relates generally to the regulation of immune responses of subjects for the diagnosis and treatment of infertility. More particularly, methods that downregulate T helper 1 (Th1) immunity or upregulate T helper 2 (Th2) immunity are used to enhance reproductive outcomes in subjects with recurrent spontaneous abortions or implantation failures by changing the balance of T helper 1 and T helper 2 immune responses. Ratios of Th1 and Th2 activities can also be used for diagnosis of infertility in these subjects.

### BACKGROUND OF THE INVENTION

Infertility is a detrimental experience in couples who want to raise a family. Infertility affects 6.1 million American women and their partners, about 10% of the reproductive age population. In 1999, about 1.2 million or 2% of reproductive age women had had an infertility related medical appointment and an additional 13% had received infertility services at some time in their lives. While many factors are reported to cause infertility, 9.7% of infertile couples received a diagnosis of unexplained infertility.

Assisted reproductive technology (ART) cycles, including in-vitro fertilization and embryo transfer (IVF/ET), gamete intrafallopian transfer and zygote intrafallopian transfer have only a 25.2% live birth rate per cycle in the U.S. Infertile women with immune etiology or multiple IVF/ET failures tend to receive a diagnosis of unexplained infertility. Implantation failure is a common reason for IVF/ET failure when pregnancy fails to occur after healthy embryos are transferred into the uterus. The ability of a normal embryo to implant itself into the endometrial lining of the uterine cavity is a pivotal event during pregnancy. Results of medical research suggest that successful implantation of the embryo depends on the proper immune response of the woman.

One proposed mechanism underlining maternal immunological tolerance of the embryo is the active immunosuppression of maternal lymphocytes. T helper lymphocytes are present at the maternal-fetal interface and may function during pregnancy. Subpopulations of T helper lymphocytes (CD3+/CD4+) can be classified as either T helper 1 (Th1) or T helper 2 (Th2) cells depending on their cytokine profiles. Th2 cells selectively produce interleukins, IL-4, IL-5, IL-6, IL-9, IL-10 and IL-13, and are involved in the development of humoral immunity against extracellular pathogens but inhibit several functions of phagocytic cells. In contrast to this, Th1 cells produce interferon-γ (IFN-γ), IL-2 and tumor necrosis factor-α (TNF-α)? and evoke cell-mediated immunity and phagocyte dependent inflammation (Mosmann & Coffman, 1989; Romagnani, 2000).

Recently, significantly higher serum levels of Th2 cytokines, IL-6 and IL-10, were detected in normal pregnancy compared to unexplained recurrent pregnancy losses and significantly higher serum levels of the Th1 cytokine, IFN-γ, were present in women with recurrent pregnancy losses compared to normal pregnancy (Raghupathy et al., 1999). These results suggested the notion that women with normal pregnancy have a Th2 bias, while women with a history of recurrent pregnancy losses have a bias toward Th1-type reactivity.

In pregnant mice, the injection of the Th1 cytokines, IFN-γ TNF-α and IL-2, increased fetal resorption (Chaouat et al., 1990). TNF-α and IFN-γ co-administration aborted over 80% of the embryos whether or not NK or macrophages had been depleted or estradiol and progesterone was injected to potential reduction in ovarian function by cytokines (Clark et al., 1998). The inventors suggest that the mechanism by which the implanted embryo is killed by Th1 cytokines is due to cytokine-triggered thrombotic/inflammatory processes at the maternal utero-placental blood vessels. The cause of abortion is ischemia due to activation of vascular endothelial cell procoagulant (Clark et al., 1998). Indeed, Th2 cytokines inhibited Th1-induced tissue factor production by monocytes (Del Prete et al., 1995). Also CD3+ and CD4+ Th1, but not Th2, T cells can help tissue factor production and procoagulant activity.

Recurrent spontaneous abortion (RSA) is a common complication of pregnancy that may affect as many as 2% of women in reproductive age (Coulam, 1991; Mills et al., 1988). Although genetic, anatomic and hormonal causes have been implicated in the etiology of RSA (Carp et al., 1990; Stray-Pedersen & Stray-Pederson, 1984), over 60% of cases remain unexplained. Various immunological abnormalities have been reported in women with RSA of unknown etiologies including autoimmune abnormalities such as positive antiphospholipid antibodies, anti-nuclear antibodies, anti-thyroglobulin antibodies and anti-microsomal antibodies, and increased cellular immunity such as elevated natural killer cell levels and NK cytotoxicity (Kwak et al., 1995; Ruiz et al., 1996b). Interestingly, these immunological abnormalities also occur in infertile women who have implantation failures after multiple IVF cycles (Beer et al., 1996; Coulam et al., 1997). These immunological similarities between women with RSA and infertility of implantation failures led us to speculate that the proclivity to Th1 cytokine responses by circulating T cells in women with RSA (Raghupathy et al., 2000) may exist in women with infertility of implantation failures. Previous studies of cytokine synthesis in peripheral blood of women with normal pregnancies or recurrent spontaneous aborters (Hill et al., 1995; Raghupathy et al., 1999) were designed to measure the total secreted cytokines from mononuclear cells but failed to discriminate the lymphocyte subpopulations. Thus studies investigating Th1/Th2 immune regulation in women with RSA or infertility of implantation failures by specifically defining the intracellular cytokine expression of CD3+/CD4+ T helper cells have not been reported.

The present invention discloses methods that downregulate T helper 1 (Th1) immunity or upregulate T helper 2 (Th2) immunity to enhance reproductive outcomes in subjects with recurrent spontaneous abortions or implantation failures by changing the balance of T helper 1 and T helper 2 immune responses. Ratios of Th1 and Th2 activities can also be used for diagnosis of infertility in these subjects.

Pharmacological chemical compounds or agents used for the treatment of infertility, early pregnancy loss, and implantation failure have been disclosed in the prior art. For example, U.S. Pat. No. 6,040,340 discloses a method for the treatment of infertility and early pregnancy loss with a nitric oxide donor alone or in combination with progesterone. However, this prior art patent does not teach the use of reduction in Th1 to Th2 ratios for the treatment of women with recurrent spontaneous abortions or implantation failures, as in the present invention. Sher et al. (American Journal of Reproductive Immunology 39(6):391-94, 1998) discloses the use of combined heparin/aspirin and IVIG immunotherapy in the treatment of recurrent IVF failure associated with antiphospholipid antibodies. This prior art publication, however, does not teach the use of reduction in Th1 to Th2 ratios for the treatment of women with recurrent spontaneous abortions or implantation failures, as in the present invention.

### SUMMARY OF THE INVENTION

The present invention provides methods for treating infertility in a subject by reducing the ratio of T helper 1 (Th1) immune response to T helper 2 (Th2) immune response in the subject to inhibit spontaneous abortion or implantation failure. In one embodiment, the Th1 immune response is the absolute cell counts of Th1 cells, and the Th2 immune response is the absolute cell counts of Th2 cells. In another embodiment, the Th1 immune response is the levels of Th1 cytokines, and the Th2 immune response is the levels of Th2 cytokines. The Th1 and Th2 cytokine levels can be serum levels or intracellular levels.

In an embodiment of the present invention, the subject is a human. In another embodiment, the subject has had one or more previous spontaneous abortions, implantation failures or IVF failures. In a preferred embodiment, the method of treating infertility in the present invention further comprises enhancing embryo implantation, pregnancy, or birth rates of the subject. In an even more preferred embodiment, the method also enhances the ability of the subject to carry at least one embryo to term. The subject may undergo natural conception, ART cycles, or ovulation induction cycles. ART includes but is not limited to in vitro fertilization.

In one embodiment of the invention, the method to reduce the ratio of Th1 response to Th2 response is to reduce the absolute counts of Th1 cells or to increase the absolute counts of Th2 cells in the subject. In an embodiment, the Th1 cells can be reduced by using an inhibitor of a costimulatory signal of a T-cell. In another embodiment, the Th2 immune response can be enhanced by administering an agent such as a Th2 immunostimulatory nucleic acid. In yet another embodiment, the method to reduce the ratio of Th1 response to Th2 response is by suppressing the levels of Th1 cytokines with Th1 cytokine antagonists or by enhancing the levels of Th2 cytokines. Preferred Th1 cytokine antagonists are TNF-A antagonists including but are not limited to infliximab, etanercept, D2E7, CDP571, CDP870, thalidomide analogs, and phosphodiesterase IV inhibitors. The Th1 cytokine antagonists can be administered by various routes such as intravenous, intramuscular, subcutaneous, transdermal, oral, vaginal, intrauterine, inhalation, mucosal and the like. The Th1 cytokine antagonists can be administered to the subject undergoing natural conception, undergoing ART cycles, or undergoing ovulation induction cycles. ART includes but is not limited to in vitro fertilization. In one embodiment, the Th1 cytokine antagonists are administered at least once prior to index conception cycle day one. In another embodiment, the antagonists are administered at least once on index conception day one. In yet another embodiment, the antagonists are administered at least once after index conception cycle day one. The subjects can also receive other treatments such as lymphocyte immunization, and/or autoimmune treatment with intravenous immunoglobulin G, anticoagulant, or an immunosuppressive agent such as prednisone.

The present invention also provides a method for diagnosing infertility in a subject with recurrent spontaneous abortions or implantation failures. The method includes the steps of determining the ratio of Th1 to Th2 immune responses of the subject and comparing the ratio to that of other subjects with normal pregnancies to determine if the subject is at risk of infertility or if the subject is suitable for treatment of the infertility by reducing the Th1 to Th2 ratios. The present invention further provides a diagnostic kit to diagnose infertility in a subject with recurrent spontaneous abortions or implantation failures. The kit consists of means of measuring Th1 and Th2 immune responses. In an embodiment, the Th1 immune response is the absolute cell counts of Th1 cells and the Th2 immune response is the absolute cell counts of Th2 cells, and the cell counts are analyzed by flow cytometry. In another embodiment, the Th1 immune response is measured by the levels of Th1 cytokines and the Th2 immune response is measured by the levels of Th2 cytokines. The Th1 and Th2 cytokines can be quantified by the use of antibodies to the cytokines. The antibodies can be poloyclonal or monoclonal antibodies or fragments thereof. In a preferred embodiment, the diagnostic kit further provides a ratio of Th1 to Th2 immune responses in a population of subjects with normal pregnancies.

The present invention further provides a method for determining whether a treatment of infertility in a subject with recurrent spontaneous abortions or implantation failures by reducing the ratio of Th1 to Th2 immune responses is having the desired effect of enhancing embryo implantation, pregnancy or birth rates in the subject by determining the ratio of the Th1 immune response to the Th2 immune response before and after the treatment to determine if the ratio is decreased.

The present invention yet further provides a method and a diagnostic kit for determining whether a TNF-α antagonist therapy will likely enhance embryo implantation, pregnancy, or birth rates in a subject by measuring the level of TNF-α in the subject and determining if the TNF-A level in the subject statistically higher than that in subjects with normal pregnancies. A preferred method of measuring the TNF-α level is by using an antibody, which can be a polyclonal or monoclonal antibody or a fragment thereof.

The present invention also provides a method and a diagnostic kit for determining whether a TNF-α antagonist treatment of infertility in a subject with recurring spontaneous abortions or implantation failures is having the desired effect of enhancing embryo implantation, pregnancy, or birth rates in the subject. The method includes the steps of measuring the levels of TNF-α in the subject before and after the treatment and determining if the TNF-α is lower after the treatment. In an embodiment, the TNF-α level is serum level. In another embodiment, the TNF-α level is intracellular level.

Additional features and advantages of the present invention are described in, and will be apparent from, the following Detailed Description of the Invention and the figures.

### BRIEF DESCRIPTION OF THE FIGURES

FIG. 1 illustrates the gating strategy for flow cytometric analysis of CD3+/CD8− (for CD3+/CD4+ analysis) and CD3+/CD8+ cells for intracellular cytokine expression;

FIG. 2 is a comparison of Th1/Th2 cytokine producing CD3+ cell ratios in women with 3 or more recurrent spontaneous abortions (RSA, n=26) and normal fertile controls (Control, n=21). Values present the mean and the standard errors;

FIG. 3 is a comparison of Th1/Th2 cytokine producing CD3+/CD8− cell ratios in women with 3 or more recurrent spontaneous abortions (RSA, n=26) and normal fertile controls (Controls, n=21); values present the mean and the standard errors; and

FIG. 4 is a comparison of Th1/Th2 cytokine producing CD3+/CD8+ cell ratios in women with 3 or more recurrent spontaneous abortions (RSA, n=26) and normal fertile controls (Controls, n=21); values present the mean and the standard errors.

### DETAILED DESCRIPTION OF THE INVENTION

While this invention is susceptible of embodiment in many different forms, there is shown in the drawing, and will be described herein in detail, specific embodiments thereof with the understanding that the present disclosure is to be considered as an exemplification of the principles of the invention and is not intended to limit the invention to the specific embodiments illustrated.

The present invention provides methods for treating and diagnosing infertility in a subject. Infertility can be treated by reducing the ratio of T helper 1 (Th1) immune response to T helper 2 (Th2) immune response in the subject to inhibit spontaneous abortion, implantation failure, which can be after ART (including but not limited to in vitro fertilization) cycles.

In an embodiment of the present invention, the subject is a human. However, the subject may also include other animal species such as domesticated animals and animals used for cloning. In another embodiment, the subject has had one or more previous spontaneous abortions or implantation failures. Implantation failures may occur after ART cycles, such as but not limited to in vitro fertilization. In a preferred embodiment, the method of treating infertility in the present invention further comprises enhancing embryo implantation, pregnancy, or birth rates of the subject. In an even preferred embodiment, the method also enhances the ability of the subject to carry at least one embryo to term. The subject may undergo natural conception, ART cycles, or ovulation induction cycles. ART includes but is not limited to in vitro fertilization.

In one embodiment, the Th1 immune response is the absolute cell counts of Th1 cells, and the Th2 immune response is the absolute cell counts of Th2 cells. What is meant by Th1 cells are the activated T-cells expressing Th1 cytokines such as IL-1, IL-2, TNF-α, and IFN-γ. Similarly, Th2 cells are the activated T-cells expressing Th2 cytokines such as IL-4, IL-5, IL-6, and IL-10. In a preferred embodiment, the Th1 cell is a TNF-α expressing CD3+/CD4+ T-cell. In another preferred embodiment, the Th2 cell is an IL-4 expressing CD3+/CD8+ T-cell. One approach of the present invention to reduce the ratio of Th1 immune response to Th2 immune response is to lower the counts of Th1 cells or to increase the counts of Th2 cells using any method known to those skilled in the art. An exemplary method to reduce counts of Th1 immune response is by the use of an agent to inhibit costimulation signal of a T-cell as disclosed in the International Pat. Application WO 01/087000, which is herein incorporated by reference and made a part hereof. Examples of such agents include but are not limited to an antibody to CD80, an antibody to CD86, and antibody to ICOS, a soluble form of CD28, and a soluble form of CTLA4. An exemplary method to increase Th2 immune response is by the use of an immunostimulatory nucleic acid as disclosed in the International Pat. Application No. WO 01/95935, which is herein incorporated by reference and made a part hereof. An example of an immuno stimulatory nucleic acid is oligonucleotides that do not contain immunostimulatory CpG motifs. Alternatively, Th2 immune response can be enhanced by lymphocyte immune therapy.

In another embodiment, the Th1 immune response is the levels of Th1 cytokines, and the Th2 immune response is the levels of Th2 cytokines. The Th1 and Th2 cytokine levels can be serum levels or intracellular levels. Examples of Th1 cytokines include but are not limited to IL-1, IL-2, TNF-α, and IFN-γ. Examples of Th2 cytokines include but are not limited to IL-4, IL-5, IL-6, and IL-10. The ratio of Th1 immune response to Th2 immune response in this embodiment is measured as a ratio of a Th1 cytokine level to a Th2 cytokine level. Various combinations of Th1 and Th2 cytokines can be used. Examples of such ratios include but are not limited to IFN-γ:IL-4; IFN-γ:IL-10; TNF-α:IL-4; and TNF-α:IL-10.

In an embodiment of the present invention, the method to reduce the ratio of Th1 to Th2 immune responses is to suppress Th1 cytokines or to enhance Th2 cytokines. Th1 cytokines can be suppressed by administering an appropriate Th1 cytokine inhibitor while Th2 cytokines can be enhanced by, for example, administering an effective dose of an appropriate Th2 cytokine such as IL-4, IL-5, IL-6, and IL-10.

Cytokine Antagonists

Substances that reduce the biological effect of a cytokine can be described as a cytokine blocker, inhibitor, or antagonist. In the present invention, the terms blocker, inhibitor, and antagonist are used interchangeably.

Cytokine antagonists can take different forms, including antibodies, receptors, and chemical compounds. Monoclonal or polyclonal antibodies for a specific cytokine can bind to the cytokine and inactivate that cytokine by prohibiting it from binding with its biological target in the human body. Alternatively, receptors for a particular cytokine can also bind to the cytokine thereby blocking its functioning. Soluble forms of receptors will be effective because they freely circulate in the body. More potent antagonists can be produced by fusing two soluble receptors together to an immunoglobulin molecule, such as an Fc fragment, and making a dimer composed of two soluble receptors with high affinity for the target and a prolonged half-life. Finally, chemical compounds may inhibit the production of a cytokine by blocking one of the steps, e.g., the step of transcription, of the biosynthesis pathway of the cytokine.

Use of Th1 cytokine antagonists has been disclosed for treatment of various immunological and inflammatory disorders. For example, U.S. Pat. No. 6,419,944 discloses the use of antagonists to TNF, IL-1, IL-6 and IL-8 for the treatment and prevention of damage to the optic nerve, other cranial nerves, spinal cord, nerve roots, peripheral nerves or muscles. This prior art patent does not teach the use of Th1 cytokine antagonists for the treatment of women with recurrent spontaneous abortions or implantation failures, as in the present invention.

U.S. Pat. No. 6,379,666 discloses the use of a TNF-α antagonist for the treatment of muscular diseases. This prior art patent does not teach the use of TNF-α antagonists for the treatment of women with recurrent spontaneous abortions or implantation failures, as in the present invention.

U.S. Pat. Nos. 6,177,077 and 6,015,557 disclose the use of a TNF-α antagonist for the treatment of inflammation and other immune response problems affecting neuronal tissues or the neuromuscular junction. These prior art patents do not teach the use of TNF-α antagonists for the treatment of women with recurrent spontaneous abortions or implantation failures, as in the present invention.

U.S. Pat. No. 5,698,195 discloses the use of a chimeric anti-TNF-α antibody for the treatment of rheumatoid arthritis. This prior art patent does not teach the use of anti-TNF-α antibodies for the treatment of women with recurrent spontaneous abortions or implantation failures, as in the present invention.

U.S. Pat. No. 5,656,272 discloses the use of a chimeric anti-TNF-α antibody for the treatment of TNF-α-mediated Crohn's disease. This prior art patent does not teach the use of anti-TNF-α antibodies for the treatment of women with recurrent spontaneous abortions or implantation failures, as in the present invention.

U.S. Pat. No. 5,919,452 discloses the use of a chimeric anti-TNF-α antibody for the treatment of a group of TNF-α-mediated diseases consisting of systemic lupus erythematosus, thyroidosis, graft versus host disease, scleroderma, diabetes mellitus, Grave's disease, sarcoidosis, chronic inflammatory bowel disease, ulcerative colitis, disseminated intravascular coagulation, atherosclerosis and Kawasaki's pathology. This prior art patent does not teach the use of anti-TNF-α antibodies for the treatment of women with recurrent spontaneous abortions or implantation failures, as in the present invention.

U.S. Pat. No. 5,385,901 discloses the use of a thalidomide analog for the control of abnormal concentration of TNF-α and for the treatment of diseases consisting of septic shock, cachexia, and HIV infection. This prior art patent does not teach the use of thalidomide analogs for the treatment of women with recurrent spontaneous abortions or implantation failures, as in the present invention.

U.S. Pat. No. 6,262,101 discloses the use of cyano and carboxy derivatives of substituted styrenes for inhibition of TNF-α and phosphodiesterase and for the treatment of diseases consisting of cachexia, endotoxic shock, retrovirus replication, asthma, and inflammatory conditions. This prior art patent does not teach the use of cyano and carboxy derivatives of substituted styrenes for the treatment of women with recurrent spontaneous abortions or implantation failures, as in the present invention.

TNF-α Antagonists

In a preferred embodiment, the method to suppress Th1 cytokines is to administer an effective dose of a TNF-α antagonist to the subject. Several TNF-α antagonists providing the possibility of therapeutic intervention are or will be commercially available. Most of these antagonists have been mainly developed to treat rheumatoid arthritis or Crohn's disease. Examples include (1) infliximab (Remicade™, sold by Centocor), a human/murine chimeric anti-TNF-o monoclonal antibody; (2) etanercept (Enbrel™, sold by Immunex), a recombinant fusion protein consisting of two soluble TNF-α receptors joined by the Fc fragment of a human IgG molecule; (3) D2E7 (also known as adalimumab or Humira, sold by Abbott Laboratories), a human anti-TNF-α monoclonal antibody; (4) CDP571 (under development by Celltech), a chimeric, but 95% humanized anti-TNF-α monoclonal antibody; (5) CDP870 (under development by Celltech), a chimeric, but 95% humanized anti-TNF-α monoclonal antibody fragment; (6) thalidomide (Thalomid, sold by Celgene), chemical compounds that suppress excessive TNF-α production; (7) structural analogs of thalidomide (IMiDS™, under development by Celgene) chemical compounds that suppress excessive TNF-α production; (8) phosphodiesterase type IV inhibitors (Selective Cytokine Inhibitory Drugs or SelCIDS™, under development by Celgene), chemical compounds that reduce the level of TNF-α; etc. When treating rheumatoid arthritis, methotrexate may be administered with infliximab to reduce the development of human anti-chimeric antibodies (HACA), since infliximab is a human/mouse chimeric monoclonal antibody and HACA may develop.

In one embodiment, infliximab can be used with the advantage of a longer interval between doses than either etanercept or D2E7. The intravenous route of administration is currently the preferred method for infliximab. The dosage range for infliximab recommended for use is from about 3 mg/Kg to about 10 mg/Kg, the same as what is recommended by the manufacturer for the treatment of rheumatoid arthritis or Crohn's disease. It is anticipated that other routes of administration of infliximab and other TNF-α antagonists can be used, which include but are not limited to subcutaneous, transdermal, vaginal, inhalation, and mucosal.

In another embodiment, etanercept is selected with the advantage of a rapid onset action, general lack of side effects, and relatively low costs. The subcutaneous route of administration is currently the preferred method for etanercept. Alternatively, the vaginal route of administration of a gel form of etanercept can be used. The preferred dosage range for etanercept recommended for use is from about 3 mg to about 50 mg. Etanercept should not be administered in a patient with an infection and its administration should be discontinued if a patient develops an infection.

In yet another embodiment, D2E7 is administered in a similar way to that of infliximab or etanercept, with the advantage of D2E7 being a fully human anti-TNF-α antibody. A preferred dosage level of D2E7 is from about 5 mg to about 50 mg, and can be administered by any acceptable routes, including but not limited to intravenous, subcutaneous, and vaginal. A more preferred dosage level of D2E7 is from about 20 mg to about 40 mg given subcutaneously once every other week.

In yet another embodiment, CDP571 or CDP870, although still in clinical development, can be used. CDP571 is a chimeric, but 95% humanized anti-TNF-α antibody; CDP870 is a fragment of the antibody. For purpose of this invention, they function in a manner similar to infliximab.

In yet another embodiment, structural analogs of thalidomide can be used. This class of compounds are immunomodulatory drugs that have been reported to be potent inhibitors of inflammatory cytokines, including TNF-α and IL-1β, while stimulating the anti-inflammatory cytokine IL-10. The preferred dosage level of thalidomide analogs is from about 50 mg/Kg to about 800 mg/Kg. Preferably, the dosage level is sufficient to produce a blood level of the thalidomide analog of at least 0.1 μg/ml. Thalidomide analogs can be administered to the subject by any acceptable routes including but not limited to oral, intravenous, subcutaneous, and vaginal. A preferred route of administration is oral.

In yet another embodiment, phosphodiesterase type IV inhibitors can reduce TNF-αproduction. Phosphodiesterase type IV inhibitors include many forms, such as amides, imides, nitriles, thalidomide analogs, hydroxamic acid derivatives, and styrene derivatives. Phosphodiesterase type IV inhibitors block TNF-α production by activating protein kinase A (PKA) and preventing transcription factors such as NF-κB from promoting transcription of the TNF-α gene.

The effective dose, route and interval of administration for each drug may vary according to individual response. The route of administration includes but are not limited to oral, vaginal, subcutaneous, and intravenous. A preferred route is vaginal administration, including formulating the agents for controlled release, such as in a gel, foam or an intrauterine device (e.g., sponge.) These agents can be administered at least once prior to, on, or after the index conception cycle day one. For infliximab, one example is a single-dose administration of 3 mg/Kg from 1 to 4 weeks prior to index conception cycle day one. Another example is a three-dose administration of 3 mg/Kg at 0, 2 and 6 weeks from index conception cycle day one. For etanercept, one example is a multiple-dose administration of from about 25 mg to about 50 mg, preferably subcutaneously, twice weekly at least four weeks prior to conception. Another example is a multiple-dose administration of a gel form 25 mg vaginally, twice weekly at least four weeks prior to conception. Additional variations of administration scheme are contemplated.

Suppressors of Other Th1 Cytokines

Antagonists of other Th1 cytokines, including antibodies, receptors of the cytokine, or agents that inhibit the biosynthesis pathway of the cytokine, can be used in the present invention. For IL-1, examples include IL-1 receptor antagonist (Kineret™, also known as anakinra, being developed by Amgen) and IL-1 receptor type II (being developed by Immunex). Other examples of Th1 cytokines include IL-2, IFN-γ, etc.

The TNF-α antagonists and other suppressors of Th1 cytokines can be administered to a subject to inhibit spontaneous abortion or implantation failure. The subject may previously have one or more spontaneous abortions or implantation failures. In a preferred embodiment, the treatment further comprises enhancing embryo implantation, pregnancy, or birth rates of the subject. In a more preferred embodiment, the treatment enhances the ability of the subject to carry at least one embryo to term. The subject may undergo natural conception or in vitro fertilization and embryo transfer. The agents may be administered at least once prior to index conception cycle day one, on index conception day one, or after index conception day one.

Methods of Diagnosis

The methods of the present invention include diagnostic methods and diagnostic kits which determine whether a woman has an imbalance of Th1 and Th2 immune responses and whether a treatment using TNF-α antagonists is likely to be effective.

In one aspect of the invention, the method is to diagnose infertility in a subject with recurrent spontaneous abortions or implantation failures. The method includes the steps of determining the ratio of Th1 and Th2 immune responses of the subject and comparing the ratio to that from subjects with normal pregnancies to determine if the subject is at risk of infertility or miscarriage or if the subject is suitable for treatment of the infertility by reducing the Th1 to Th2 ratios. The Th1 and Th2 immune response may be determined by methods such as flow cytometry analysis by measuring the counts of Th1 and Th2 cytokine expressing T-cells. An example of suitable Th1 cells is the TNF-α expressing CD3+/CD4+ T-cells. An example of suitable Th2 cells is the IL-4 expressing CD3+/CD4+ T-cells. Alternatively, the Th1 and Th2 immune response may be determined by the levels of serum, culture supernatant or intracellular Th1 and Th2 cytokines. Examples of suitable Th1 and Th2 cytokines are discussed previously. Various combinations of Th1 and Th2 cytokines can be used. Examples of such ratios include but are not limited to IFN-γ:IL-4; IFN-γ:IL-10; TNF-α:IL-4; and TNF-α:IL-10.

A diagnostic kit for the diagnostic method would comprise means for determining Th1 immune response and means for determining Th2 immune response. In one embodiment, the Th1 immune response is the levels of Th1 cytokines in the subject, the means for determining the Th1 immune response comprises a Th1 cytokine antibody, the Th2 immune response is the levels of Th2 cytokines in the subject, and the means for determining the Th2 immune response comprises a Th2 cytokine antibody. The antibody can be a polyclonal or monoclonal antibody or a fragment thereof. In a preferred embodiment, the diagnostic kit further provides a ratio of Th1 to Th2 immune responses in a population of other subjects with normal pregnancies. The ratio of Th1 to Th2 of the subject can be compared to that of the normal subjects

In another aspect of the invention, the diagnostic method determines whether a treatment of infertility in a subject with recurrent abortions or implantation failures by reducing the ratio of Th1 to Th2 immune responses is having the desired effect of enhancing embryo implantation, pregnancy, or birth rates. The method includes the steps of determining the ratio of Th1 to Th2 immune responses of the subject before and after the treatment and determining if the ratio is reduced after the treatment to determine if the treatment has the desired effect of enhancing embryo implantation, pregnancy, or birth rates.

In yet another aspect of the invention, the diagnostic method determines whether TNF-α antagonist will likely enhance embryo implantation, pregnancy, or birth rates in a subject with recurrent spontaneous abortions or implantation failures. The method includes the steps of measuring the level of TNF-α in the subject to determine if the level is statistically higher than that in subjects with normal pregnancies. In one embodiment, subjects with serum TNF-α levels higher than 12 pg/ml with a history of two or more spontaneous abortion or IVF/ET implantation failures may be selected for treatment. Serum TNF-α can be measured by any method known to those skilled in the art. An example of such a method is the use of an antibody against TNF-α. The antibody can be a polyclonal or monoclonal antibody or a fragment thereof. Alternatively, intracelluar TNF-α expression, e.g., in CD3+/CD4+ cells, may be measured by flow cytometry and compared with data in women with normal pregnancies. A diagnostic kit for this test may comprise means for measuring the levels of TNF-α, such as an antibody against TNF-α. The antibody can be a polyclonal or monoclonal antibody or a fragment thereof.

In a further aspect of the invention, the diagnostic method determines whether TNF-α antagonist treatment of infertility in a subject with recurring spontaneous abortions or implantation failures is having the desired effect of enhancing embryo implantation, pregnancy, or birth rates in the subject. The method includes the steps of measuring the levels of serum or intracelluar TNF-α levels in the subject before and after the treatment to determine if the level of TNF-α is decreased after the treatment to determine if the treatment is having the desired effect of enhancing embryo implantation, pregnancy, or birth rates in the subject.

It is contemplated that the above methods can be applied before, during and/or after the treatment of a woman and the results can be used to determine whether treatment of a woman with recurrent spontaneous abortions or implantation failures by suppressing Th1 immunity and/or enhancing Th2 immunity is having the desired effect.

Combinations of Methods or Techniques

The methods of the present invention can be used alone or can be used with other techniques or methods. For example, certain patients receiving a TNF-α antagonist therapy may receive additional therapeutic benefit from the use of lymphocyte immune therapy to increase Th2 immune response in addition to the use of a TNF-α antagonist. The combination produces a more potent anti-inflammatory effect than when either is administered alone. Other examples of techniques or methods that can be used together with methods of the present invention include administration of an immunosuppressive agent (e.g., prednisone), intravenous immunoglobulin G (IVIg), anticoagulants (heparin and/or aspirin), etc.

### EXAMPLE 1

### Th1/Th2 Ratios in Women with RSA and Infertility Population

The study design was a prospective controlled study. Study groups and controls were enrolled consecutively if they met the study inclusion criteria. The study was performed at the Division of Reproductive Medicine, Department of Microbiology and Immunology, Finch University of Health Sciences/The Chicago Medical School. All the study and control subjects had signed an informed consent prior to entering the study. Blood was drawn prior to any treatment including IVF cycles. No one was on any medication.

Inclusion criteria for this study are; 1) fertile women with 3 or more recurrent pregnancy losses of unknown etiology or 2) infertile women with 2 or more implantation failures after IVF cycles, who had 2 or more embryos transferred per each IVF cycle, excluding donor egg cycles; 3) not pregnant; 4) no more than 1 live born infant; 5) age ranges 25-45 years old; and 6) no active disease including autoimmune disease. To investigate a possible medical condition, physical examination, past history review, review of system and blood tests were performed. Blood tests are comprehensive metabolic panel, complete blood count, thyroid function test (Free T4 and TSH), antinuclear antibody, antibodies to single stranded DNA, double stranded DNA and histone.

The recurrent abortion group included 26 women with 3 or more RSA of unknown etiology with the same partner. Three had one child. All had pregnancy losses during the first trimester of gestation. None had active autoimmune disease or a history of autoimmune disease. No one with evidence of active autoimmune disease was included in this study. No apparent causes of recurrent abortion such as chromosomal, endocrine, anatomical, or infectious etiologies were documented for previous pregnancy losses. None had infertility or received IVF cycles. Table I lists age, obstetrical histories and IVF histories of RSA groups and normal fertile controls. Age distribution is comparable between women with RSA and normal fertile controls.

TABLE I
Age and obstetrical histories of women with 3 or more recurrent
spontaneous abortions (RSA), and normal fertile controls.

| | Controls (n = 21) | RSA (n = 26) | |
|---|---|---|---|
| | (Mean ± SD) | (Mean ± SD) | P value |
| Age | 38.3 ± 8.0 | 34.4 ± 5.6 | NS |
| Gravidity | 2.6 ± 0.9 | 4.6 ± 2.0 | <0.05 |
| SAB* | 0.0 ± 0.0 | 4.3 ± 1.7 | <0.000 |
| IVF/ET failure | 0.0 ± 0.0 | 0.0 ± 0.0 | NS |

*SAB; spontaneous abortions

Total 23 women of infertility with implantation failures after 2 or more IVF cycles comprised the implantation failure groups. Based on their history of spontaneous abortion (SAB), women with implantation failures were divided to two subgroups. Multiple IVF failures without SAB group had fourteen women. None had a history of spontaneous abortion and two have one live child (One had 8 IVF failures and the other had 5 IVF failures). Multiple IVF failures with SAB group included 9 women. Two had one live child (One had 6 IVF failures and the other had 3 IVF failures). In women with multiple IVF failures, no one had active autoimmune disease or a history of autoimmune disease. No apparent cause for IVF failures has been documented in these women. Women with male factor infertility and donor egg cycles were excluded. Table II lists age, obstetrical histories, IVF histories including their primary infertility diagnosis.

TABLE II
Age, obstetrical and in-vitro fertilization cycle histories of
infertile women with multiple implantation failures after 2 or
more IVF cycles with and without a history of spontaneous
abortion (SAB).

| | Infertility (n = 23) | | |
|---|---|---|---|
| | (Mean ± SD) | | |
| | No history of SAB | History of SAB | |
| | (n = 14) | (n = 9) | P value^{§} |
| Age | 36.9 ± 4.6 | 35.2 ± 4.6 | NS |
| Gravidity | 0.5 ± 0.6 | 2.8 ± 1.1 | <0.000 |
| SAB | 0.0 ± 0.0 | 2.5 ± 1.1 | <0.000 |
| No. of failed IVF | 4.3 ± 1.9 | 4.5 ± 1.6 | NS |
| cycles | | | |
| Number of mature | 9.1 ± 5.0 | 15.3 ± 5.9 | 0.002 |
| oocytes/cycle | | | |
| Number of eggs fertil- | 5.5 ± 4.1 | 9.4 ± 6.7 | NS |
| ized/cycle | | | |
| Number of embryos | 3.1 ± 1.6 | 3.6 ± 1.5 | NS |
| transferred/cycle | | | |
| Primary infertility Dx. | | | NS* |
| Endometriosis (No.) | 2 | 1 | |
| Tubal factor (No.) | 5 | 2 | |
| Unexplained (No.) | 7 | 6 | |

^{§}Two sample two tailed t-test was applied except the primary infertility diagnosis.
*Chi-square analysis was applied.

Controls were 21 normal multiparous healthy non-pregnant women with documented uncomplicated pregnancies (≧1, ranges 1-3). All controls were interviewed, during which time personal and family histories were ascertained. None had an active disease including autoimmune disorder. All had a history of one or more normal deliveries. None had a history of pregnancy loss, infertility, or implantation failures (Table I).

Cell Separation and Culture

Peripheral blood mononuclear cells (PBMC) were isolated by Ficoll-Hypaque (Biotech, Uppsala, Sweden) density centrifugation. After washing in RPMI 1640 (Gibco-BRL, Life-Technology N.Y. USA), the cells were adjusted to a concentration of 1×10⁶/ml in RPMI 1640 supplemented with 10% fetal bovine serum (Gibco-BRL, Life-Technology, N.Y., U.S.A.), 100 U/ml penicillin, 100 μg/ml streptomycin, 0.25 μg/ml amphotericin B (PSA-100× reagent from Sigma, St. Louis, USA). To activate the PBMC 1 ml of the cell suspension was incubated with phorbol myristate acetate (PMA) (Sigma, St. Louis, Mo.) at final concentration of 25 ng/ml, and ionomycin (Sigma, St. Louis, Mo.), 1 μM, for 5 hours at 37° C. in a humidified incubator in 5% CO2. Monensin (Pharmingen, SanDiego, Calif.) at 2 μM was also added at the start of culture to inhibit cytokine secretion.

Preparation of Cells for Flow Cytometric Analysis

The cells were washed in PBS with 1% heat-inactivated FBS and 0.09% sodium azide (staining buffer). Then fluorochrome-conjugated monoclonal antibodies specific for cell surface antigens were incubated with the cells for 15 minutes at room temperature. The combinations were as follows: 10 μl each of anti-CD3-ECD and anti-CD8-FITC to identify the two cell populations, CD3+CD8+ and CD3+CD8−, or anti-CD69-PC5 to determine the percentage of activation following stimulation for five hours. The cytokine expression levels were undetectable without stimulation after 5 hours of culture with monensin only (Pharmigen, San Diego, Calif.). Due to the undetectable cytokine expression without stimulation, cells were stimulated using phorbor 12-myristate 13-acetate (PMA) and ionomycin. Less than 2% of CD3+ cells demonstrated CD69 expression without stimulation. It has been shown that following stimulation of lymphocytes with PMA and ionomycin, a rapid down regulation of CD4 molecules on the surface of lymphocytes occurs. In applicants' hands, a decrease of CD4 occurred as rapidly as after 4 hours of stimulation. Therefore, a negative gating strategy was used to measure intracellular cytokine expression in CD3+CD4+ cells. Cells are reacted with anti-CD3 and anti-CD8 and cells that are CD3+ but not CD8+ are considered to be CD3+CD4+ cells (Rostaing et al., 1999).

After two more washes with staining buffer, the cell pellet was fixed and permeabilized for 20 minutes using 250 μl of cytofix/cytoperm solution (Pharmingen, San Diego, Calif.) according to the manufacturer's instructions. Afterward the cells were washed twice and resuspended in 50 μl of 1×penn/wash solution (Pharmingen, San Diego, Calif.) containing a predetermined optimal concentration of PE-conjugated anti-cytokine antibodies for 30 minutes at room temperature. The concentrations were usually <0.2 μg mAb/million cells for IL-4, and 0.5 μg mAb/million cells for IFN-γ, TNF-α, and IL-10). For each patient the appropriate isotype control was also used. After staining, the cells were washed twice with 1×penn/wash solution and resuspended in 0.5 ml of staining buffer. The cells were stored at 4° C. until analysis by flow cytometry within 24 hours. In this study, Th2 cytokine (IL-4 and IL-10) producing cells were relatively low. Although the same pattern has been reported previously (Tsuda & Yamasaki, 2000), applicants determined to verify IL-10 and IL-4 data by analyzing isotype controls. Non-specific intracellular staining with isotype control antibodies was 0.19±0.15% for IL-10 expression study and 0.17+0.13% for IL-4 expression study.

Reagents and Antibodies

The final concentrations of the stimulants used in cell culture were as follows: phorbol myristate acetate (PMA: Sigma, St. Louis, USA) 25 ng/ml, ionomycin (Sigma, St. Louis, USA) 1 μM, monensin (Pharmingen, SanDiego, Calif.) 2 μM. Antibodies that were used were as follows: anti-cytokine antibodies were PE-anti-human IFN-γ, clone 4S.B3; PE-anti-human TNF-α, clone Mab11; PE-anti-human IL-4, clone 8D4-8; PE-anti-human IL-10, clone JES3-19F1; PE-mouse IgG1 isotype, clone MOPC-21; PE-rat IgG2 isotype, clone R35-95 (Pharmingen, San Diego, U.S.A.). Antibodies to lymphocytes were: FITC-anti-human CD8, clone T8; ECD-anti-human CD3 (Beckman-Coulter, Miami, Fla.), PC5 anti-human CD69, clone TP1.55.3 (Beckman-Coulter, Miami, Fla.).

Acquisition and Analysis of Flow Cytometric Data

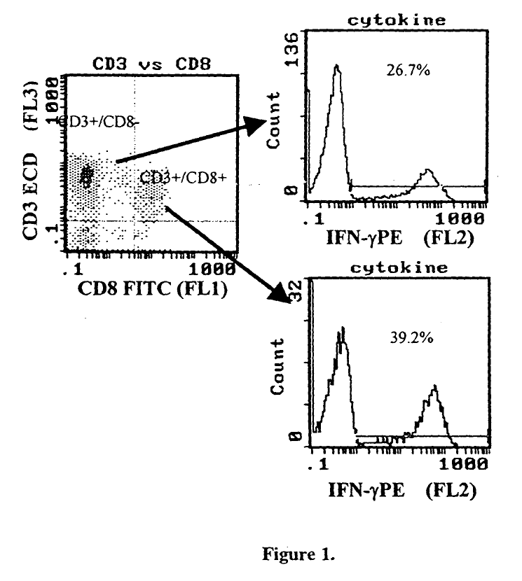

The stained and fixed samples were analyzed on a Coulter XL flow cytometer using XL software. Forward vs. side scatter events was acquired to analyze the lymphocyte population. Fluorescence from the FL1 (FITC), FL2 (PE), FL3 (ECD) and FL4 (PC5) channels were used to measure cell surface and intracellular fluorescence. Compensations were determined on the lymphocyte gate using tubes with singularly labeled strongly positive antibodies. The number of events acquired for each sample was 40,000. A region based on light scatter (FALC vs. SS) was drawn around the major lymphocyte population. This population was used to obtain the gated fluorescence plot of CD3 versus CD8. Rectangular regions were set to include all the CD3+CD8+ bright cells and all the CD3+CD8−(CD3+/CD4+) bright cells obtained by negative gating strategy (FIG. 1). All data are expressed as the percentage of cytokine-positive CD3+/CD8− or CD3+/CD8+ bright cells. A cell surface activation antigen (CD69-FITC) was also used on these permeabilized and fixed cells to show activation status of the CD3+/CD8− (CD3+/CD4+) and CD3+/CD8+ cells within the tight lymphocyte light scatter gate used for the analysis of intracellular cytokines. FIG. 1 illustrates the gating strategy for flow cytometric analysis of CD3+/CD8− (for CD3+/CD4+ analysis) and CD3+/CD8+ cells for intracellular cytokine expression.

Statistical Analysis

The statistical analysis was performed using SPSS/PC+ TM program. Since women with RSA do not have any infertility or implantation failure histories, two separate statistical analyses were performed; 1) The study results of women with RSA were compared to those of normal fertile controls, and 2) The study results of infertile women with multiple implantation failures with SAB, without SAB and normal fertile controls were analyzed.

Unpaired two tailed t-test was applied for comparisons of intracellular cytokine expression and proportion of T cell subsets between women with RSA and normal fertile controls. Since the statistical analysis is applied to investigate if the elevated Th1:Th2 ratios in CD3+, CD3+/CD8− and CD3+/CD8+ cells of women with RSA are significantly different as compared to those of normal fertile controls, unpaired one tailed t-test was applied. If the population variances in the two groups are equal, the pooled-variance t-test was applied. If the population variances in the two groups are different, the separate variance t-test was applied. Differences were considered significant, if p value is equal or less than 0.05.

For the comparison of intracellular cytokine expression, T cell subsets and the Th1:Th2 ratios among women with multiple implantation failures without SAB, with SAB and normal fertile controls, one way analysis of variance with the Scheffe multiple comparison test was applied. The Scheffe multiple comparison test was applied to protect against calling too many differences being significant. This test allows more stringent criteria for declaring differences significant than usual t-test.

T Cell Subsets and Activation Status

Peripheral blood T lymphocyte subpopulations were determined by flow cytometric analysis. There was no statistically significant difference in the proportion (%) of CD3+, CD3+/CD8− and CD3+/CD8+ cells between women with RSA and normal fertile controls, and women with infertility of implantation failures without SAB, with SAB and normal fertile controls (Table III).

TABLE III
CD3+, CD3+/CD8− (T helper) and CD3+/CD8+ (T suppressor)
subsets in women with 3 or more recurrent abortions (RSA),
multiple implantation failures after 2 or more IVF cycles with and
without a history of spontaneous abortion (SAB) and normal
fertile controls.

| | | | IVF failures | |
|---|---|---|---|---|
| | | | (n = 23) | |
| | | | No history of | |
| | Controls | RSA | SAB | History of SAB |
| T cell | (n = 21) | (n = 26) | (n = 14) | (n = 9) |
| subsets | Mean ± SD^{a} | Mean ± SD | Mean ± SD | Mean ± SD |
| CD3+ | 72.8 ± 10.2 | 72.3 ± 10.7 | 74.5 ± 9.4 | 75.3 ± 7.6 |
| CD3+/ | 52.5 ± 9.5 | 53.0 ± 8.8 | 53.7 ± 6.6 | 55.4 ± 7.2 |
| CD8− | | | | |
| CD3+/ | 20.3 ± 6.6 | 19.2 ± 5.0 | 20.8 ± 6.0 | 19.9 ± 5.0 |
| CD8+ | | | | |

P = NS; Comparisons made between RSA vs Controls, and in IVF failures without SAB, with SAB and controls.
^{a}SD, standard deviation.

To measure intracellular cytokines, resting lymphocytes must first be activated for 5 hours with PMA and ionomycin. For the confirmation of lymphocyte activation, CD69 expression on T cell sub-populations was measured in study and control subjects. 88.2±4.3% (Mean±SD) cells of women with RSA, 88.8±4.1% cells of women with infertility of implantation failures without SAB, 88.6±4.0% cells of women with infertility of implantation failures with SAB, and 87.4±6.0% of normal fertile controls were activated as judged by the expression of the early activation antigen CD69 after 5-hour incubation with PMA and ionomycin. The proportions of activated cells (CD69+) in study and control groups were not statistically different. These results assured that lymphocyte stimulation with PMA and ionomycin was comparable in study and control groups.

Intracellular Cytokine Expression

The proportion (%) of TNF-α, IFN-γ, IL-4 and IL-10 producing CD3+, CD3+/CD8−, CD3+/CD8+ cells in women with RSA, infertile women with multiple implantation failures without a history of SAB, with a history of SAB and normal fertile controls are listed in Table IV. The proportion of IL-10 producing CD3+/CD8+ cells was significantly higher in women with RSA as compared to that of normal fertile controls (P=0.013). The proportion of TNF-α producing CD3+/CD8− cells was significantly different among women with implantation failures without SAB, with SAB and normal fertile controls (P=0.011).

TABLE IV
Intracellular cytokine expression in CD3+, CD3+/CD8− (T helper) and
CD3+/CD8+ (T suppressor) lymphocytes in women with recurrent
spontaneous abortion (RSA), multiple implantation failures after 2 or
more IVF cycles with and without a history of spontaneous abortion
(SAB) and normal fertile controls.

| | | | | IVF failures | | |
|---|---|---|---|---|---|---|
| | | | | (n = 23) | | |
| | Controls | RSA | | No history of SAB | History of SAB | |
| | (n = 21) | (n = 26) | | (n = 14) | (n = 9) | |
| Cytokine | Mean ± SD^{a} | Mean ± SD | P value⁺ | Mean ± SD | Mean ± SD | P value^{§} |

CD3+ T cells

| | | | | | | |
|---|---|---|---|---|---|---|
| IFN-γ | 19.40 ± 8.31 | 18.79 ± 8.78 | NS | 20.26 ± 9.63 | 18.62 ± 4.26 | NS^{b} |
| TNF-α | 24.61 ± 10.23 | 25.50 ± 11.61 | NS | 33.42 ± 12.94 | 26.37 ± 8.67 | NS |
| IL-4 | 2.46 ± 0.78 | 2.14 ± 0.95 | NS | 2.69 ± 1.62 | 2.42 ± 1.07 | NS |
| IL-10 | 0.95 ± 0.41 | 0.75 ± 0.33 | NS | 0.77 ± 0.33 | 0.69 ± 0.22 | NS |

CD3+CD8− T helper cells

| | | | | | | |
|---|---|---|---|---|---|---|
| IFN-γ | 14.23 ± 6.52 | 14.47 ± 6.22 | NS | 17.59 ± 9.05 | 14.92 ± 5.42 | NS |
| TNF-α | 24.80 ± 10.23 | 27.0 ± 12.85 | NS | 38.29 ± 16.42* | 28.36 ± 8.99 | 0.011 |
| IL-4 | 2.75 ± 0.93 | 2.36 ± 1.04 | NS | 2.67 ± 0.97 | 2.53 ± 0.77 | NS |
| IL-10 | 0.90 ± 0.41 | 0.75 ± 0.36 | NS | 0.69 ± 0.21 | 0.68 ± 0.23 | NS |

CD3+/CD8+ T suppressor

| | | | | | | |
|---|---|---|---|---|---|---|
| IFN-γ | 34.82 ± 13.34 | 31.97 ± 17.20 | NS | 29.23 ± 13.97 | 30.64 ± 13.46 | NS |
| TNF-α | 24.73 ± 13.19 | 21.83 ± 12.08 | NS | 22.92 ± 14.11 | 21.84 ± 13.33 | NS |
| IL-4 | 2.04 ± 1.18 | 1.63 ± 1.07 | NS | 1.47 ± 1.06 | 1.45 ± 0.87 | NS |
| IL-10 | 1.20 ± 0.59 | 0.83 ± 0.39 | 0.013 | 1.00 ± 0.78 | 0.69 ± 0.26 | NS |

⁺Comparisons were made between women with RSA and normal fertile controls using two-tailed t-test.
^{§}Comparisons were made using one way analysis of variance with the Scheffé multiple comparison test in women with IVF failures without SAB, with SAB and normal fertile controls.
*P < 0.05 as compared to controls by the Scheffé multiple comparison test.
^{a}SD, standard deviation.
^{b}NS, not significant

Th1/Th2 Cytokine Ratios

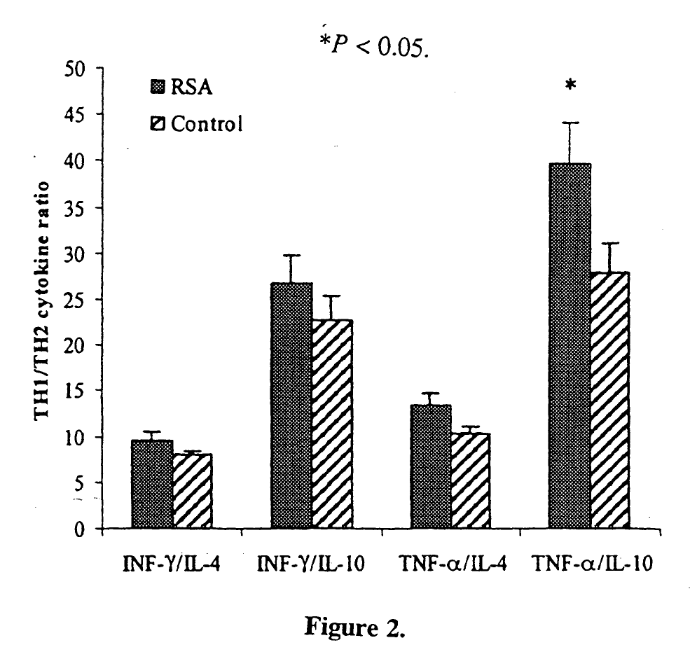

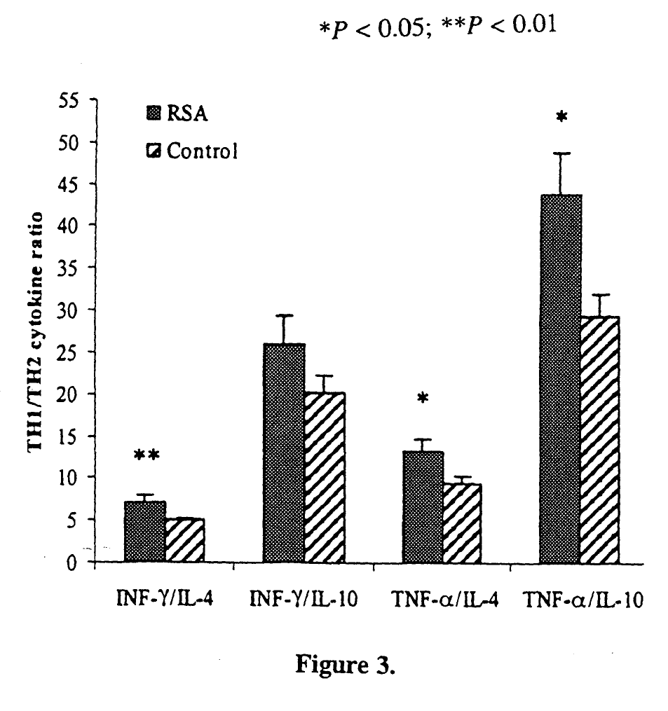

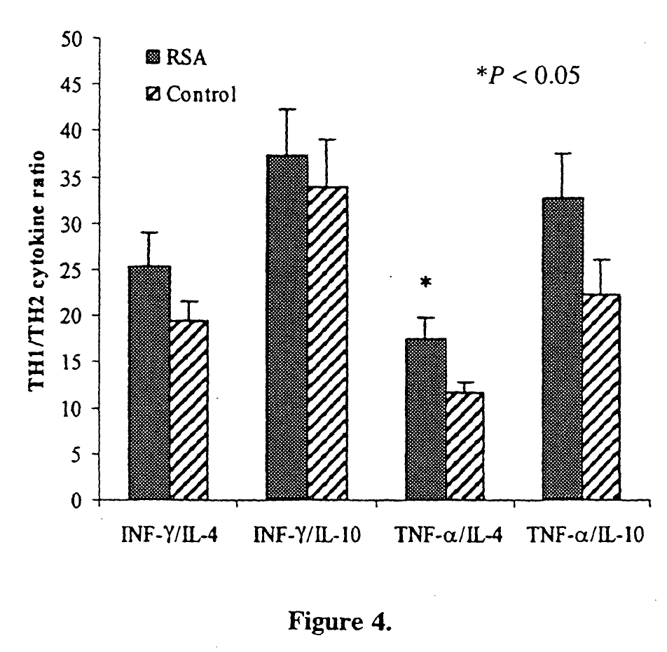

To compare the proportion of T cell subsets synthesizing Th1 cytokines versus Th2 cytokines in each woman, the ratios of Th1/Th2 were calculated by dividing the proportion of Th1 cytokine producing cells by the proportion of Th2 cytokine producing cells with the following combination; IFN-γ/IL-4, IFN-γ/IL-10, TNF-α/IL-4 and TNF-α/IL-10. After calculating each study subject's Th1/Th2 ratios, the mean and standard error of each study group was calculated. FIG. 2 (CD3+ cells), FIG. 3 (CD3+/CD8− cells) and FIG. 4 (CD3+/CD8+ cells) plot the Th1/Th2 cytokine ratio of women with RSA and normal fertile controls. The results are shown in FIGS. 2 to 4. Table V demonstrates the Th1/Th2 cytokine ratios of women with multiple implantation failures after IVF cycles without SAB, with SAB and normal fertile controls.

TABLE V
The ratios of Th1/Th2 related intracellular cytokine expression in CD3+,
CD3+/CD8− (T helper), and CD3+/CD8+ (T suppressor) lymphocytes in
women with 2 or more IVF failures and normal fertile controls.

| | | | IVF failures | | |
|---|---|---|---|---|---|
| | | | (n = 23) | | |
| | | Controls | No history of SAB^{a} | History of SAB | |
| | | (n = 21) | (n = 14) | (n = 9) | |
| T cellsubsets | Cytokine ratio | Mean ± SE^{a} | Mean ± SE | Mean ± SE | P Value^{§} |
| CD3+ | IFN-γ/IL-4 | 7.94 ± 0.46 | 8.61 ± 1.10 | 9.14 ± 1.49 | NS^{b} |
| | IFN-γ/IL-10 | 22.67 ± 2.73 | 29.55 ± 3.67 | 29.68 ± 4.34 | NS |
| | TNF-α/IL-4 | 10.31 ± 0.80 | 15.88 ± 2.17 | 13.14 ± 2.80 | 0.050 |
| | TNF-α/IL-10 | 27.88 ± 3.27 | 46.37 ± 6.84* | 42.57 ± 7.73 | 0.030 |
| CD3+/CD8− | IFN-γ/IL-4 | 5.02 ± 0.29 | 6.74 ± 0.75 | 6.33 ± 0.90 | NS |
| (T helper) | IFN-γ/IL-10 | 20.05 ± 2.29 | 26.55 ± 3.29 | 26.03 ± 6.06 | NS |
| | TNF-α/IL-4 | 9.49 ± 0.79 | 15.96 ± 2.30* | 12.81 ± 2.52 | 0.028 |
| | TNF-α/IL-10 | 29.45 ± 2.60 | 60.05 ± 8.63** | 48.67 ± 10.08 | 0.0043 |
| CD3+/CD8+ | IFN-γ/IL-4 | 19.55 ± 1.94 | 25.22 ± 4.23 | 23.29 ± 4.22 | NS |
| (T suppressor) | IFN-γ/IL-10 | 34.04 ± 5.01 | 39.87 ± 7.52 | 51.17 ± 9.34 | NS |
| | TNF-α/IL-4 | 11.61 ± 1.27 | 20.32 ± 4.16 | 17.48 ± 4.10 | NS |
| | TNF-α/IL-10 | 22.42 ± 3.65 | 44.06 ± 10.12 | 40.56 ± 8.75 | 0.049 |

^{§}Comparisons were made using one way analysis of variance with the Scheffé multiple comparison test.
*P < 0.05 as compared to controls by the Scheffé multiple comparison test.
**P < 0.01 as compared to controls by the Scheffé multiple comparison test.
^{a}SE, standard error.
^{b}NS, not significant

Both animal models and studies in humans have suggested that pregnancy benefits from Th2 cytokines and can be threatened by Th1 cytokines (Chaouat et al., 1995; Raghupathy et al., 2000). The induction of Th1 responses produced by injection of IL-2, TNF-α and IFN-γ, can cause spontaneous abortion in a murine model (Chaouat et al., 1990). In contrast, intraperitoneal rIL-10 injection reverses the high incidence of fetal resorption and either anti-IFN-gamma or pentoxifillin (an anti-TNF agent) partially reduces the fetal resorption in mice with resorption prone CBA×DBA/2 matings (Chaouat et al., 1995). In women with recurrent pregnancy losses, immunoglobulin G infusion treatment (Ruiz et al., 1996a) and lymphocyte immunotherapy (Kwak et al., 1998) reduce NK cytotoxic activity and NK cell levels, and favors a successful pregnancy (Group, 1994; Kwak et al., 1996). This is consistent with the observation that cytotoxic activities induced by Th1 cytokines have deleterious effects on pregnancy and that the prevention of such responses protects pregnancy.

In this study, applicants report that intracellular Th1 cytokine expressions are increased over Th2 cytokine expressions in women with RSAs and infertility of multiple implantation failures. This study is consistent with the previous reports of increased Th1 immune responses in women with recurrent pregnancy losses (Lim et al., 2000; Raghupathy, 1997a). This proclivity to Th1 cytokine responses by T cells is mainly expressed in CD3+/CD8− cells, but also in CD3+/CD8+ cells in women with recurrent pregnancy losses and infertile women with multiple implantation failure after IVF cycles. These results were obtained by analysis of peripheral blood lymphocytes that reflect systemic expression and regulation. Indeed applicants found the ratio of Th1:Th2 immune responses is more important than the expression of a single cytokine.

Recent advances in immunoassays such as commercially available standardized cell permeabilization reagents rather than saponin, antibodies to cytokines which are directly conjugated to give low background fluorescence, and the use of different combinations of monoclonal antibodies for four color flow cytometry have led to greater flexibility and more consistency in determining intracellular cytokine expression (North et al., 1996). In this study applicants investigated CD3+/CD4+ Th1 and Th2 cells by measuring CD3+/CD8− cells based on intracellular cytokine expression and also evaluated cytokine expression in CD3+/CD8+ cells in women with RSA or infertility of implantation failures. NK cells, which are CD3−, were not included in the flow cytometric analysis. A concern may be raised that CD3+/CD8− cells are measured for CD3+/CD4+ cells. These two cell populations are probably identical, although by definition of expression they are not.

In this study, lymphocytes were stimulated with PMA and ionomycin in the presence of a protein transport inhibitor monensin to allow cytokines to accumulate in levels that were high enough to be detected by intracellular flow cytometric analysis. Applicants have demonstrated the prevalence of Th1 immune responses over Th2 immune responses in specific T cell subpopulations based on expression of each cytokine. Previous in vitro studies utilized trophoblast antigens to activate lymphocytes of women with a history of RSA and reported the presence of cytokines in the supernatant that was injurious to the developing conceptus or trophoblast cell lines (Ecker et al., 1993; Hill et al., 1995). This study suggests that altered Th1 immune responses can also be detected systemically as a result of an in vivo challenge during pregnancy (RSA) or in-vitro fertilization cycles.

TNF-α is supposed to suppress the growth of trophoblasts (Todt et al., 1996), possibly by inducing apoptotic changes in these cells (Yui et al., 1994). TNF-α is present on the proliferating tips of anchoring villi, invasive interstitial cytotrophoblasts, and endovascular trophoblasts which invade spiral arteries (Lea et al., 1997). These findings suggest a role for TNF-α in early invasion of trophoblasts. However, a decrease in the release of TNF-α from PBMCs upon the recognition of HLA-G was a consistent finding among normal women, recurrent aborters, and men (Maejima et al., 1997). Perhaps, the regulation of TNF-α synthesis may determine reproductive outcome. In this study, TNF-α expression in CD3+/CD8− cells from infertile women with implantation failures is also significantly up-regulated as compared with that of normal controls. More importantly, the ratio of TNF-α to IL-4 or IL-10 expressing cells are persistently elevated in both women with RSA and infertility of implantation failures as compared to those of normal fertile controls, and infertile women with multiple implantation failures without SAB demonstrated the highest Th1/Th2 ratios particularly related with TNF-α related ratios. These findings are examples of the relationship between TNF-α, implantation and pregnancy outcome.

IL-10 is known to selectively suppress Th1 mediated cellular immunity by inhibiting the production of inflammatory cytokines such as IFN-γ, TNF-α, and IL-1 (Mosmann & Moore, 1991). Decreased production of IL-4 and IL-10 by decidual T cells of women with unexplained RSA when compared to decidual cells of women with normal pregnancy has been reported (Piccini et al., 1998). This study demonstrated a significant difference in IL-10 expression in activated peripheral blood CD3+/CD8+ cells in recurrent aborters.

In the mouse model, it has been suggested that placental antigens from resorption-prone CBAxDBA/2 mating activate CD8+ T cells, which result in abortion, but placental antigens from non-resorption prone mating fail to activate CD8+ T cells (Raghupathy, 1997b). In vivo injection of anti-CD8+ T cells into abortion-prone CBA/J×DBA/2 pregnancies either has no effect on the abortion rate or boosts the abortion rate depends on days of gestation (Chaouat & Menu, 1997). CD8+ T cells have apparent opposing effects, which may in part be explained by the Th1/Th2 paradigm and the fact that CD8+ T cells can potentially belong to either phenotype. In this study, TNF-α/IL-10 ratios in CD3+/CD8+ T cells were significantly different in women with infertility of implantation failures as compared to those of normal controls. Applicants' collateral study using the same population of patients demonstrated significantly elevated activated NK cells (CD56+/CD69+) in peripheral blood (Ntrivalas et al., 2001). The presence of activated NK cells in these women may be related to the activation status of CD3+/CD8+ cells, which have decreased Th2 cytokine production.

The underlying etiology of Th1/Th2 polarization needs further investigation. While applicants do not wish to be bound by any theoretical mechanisms, it is possible that the Th1 shift may be mediated by T cells or antigen presenting cells that direct the differentiation of effector cells. From this study, it is interesting to notice that lymphocytes from women with implantation failures, who had never become pregnant and have had no chance to be exposed to trophoblasts antigens, or never had any history of pregnancy losses demonstrated an increased Th1 shift in their cytokine expression. Therefore, it is plausible that increased synthesis of Th1 cytokines may be induced not only by trophoblast antigens, but also by antigen-nonspecific cytokine/chemokine production in response to stress products of hormonally manipulated endometrium, hyperstimulated ovarian products or non-physiological high levels of female sex hormones.

A question was raised if women with implantation failure without a history of spontaneous pregnancy losses have Th1 shift. In this study, women with implantation failures without a history of SAB were compared to women with implantation failures with a history of SAB and normal fertile controls. This study setting allows applicants to investigate the impact of pregnancy losses and Th1 shift in regards to implantation failures. This study demonstrated that women with implantation failures without any history of SAB have the highest Th1 shift, women with implantation failures with a history of SAB have the 2^{nd} highest, then women with RSA have the 3^{rd} highest Th1 shift as compared to normal fertile women. Interestingly, infertile women with multiple implantation failures without a history of SAB demonstrated significantly lower number of mature eggs than infertile women with multiple implantation failures with SAB history in their previous IVF cycles. Whether the ovarian stimulation response is affected by Th1/Th2 shift is subject to study.

Since the introduction of IVF/ET technique, the implantation rate after IVF cycles has not dramatically increased. Prenatal genetic diagnosis may explain this low implantation rate in part (Kahraman et al., 2000). However, this study also raises an important question for the role of Th1/Th2 immune responses in failure of embryonic implantation. Further study is needed to explore the relation between T helper cell cytokine regulations, MHC complex, and reproductive outcome in women with altered Th1 immune responses.

### EXAMPLE 2

### Treatment of Infertility by the Use of Infliximab

11 women with multiple implantation failures after IVF/ET cycles and elevated TNF-α levels were selected for infliximab therapy. All 11 women satisfied the following criteria: (1) infertility of unknown etiology; (2) two or more IVF failures; (3) age 40 years or less; (4) elevated serum TNF-α of 12 pg/ml (the normal TNF-α level is 0-12 pg/ml); (5) no viable pregnancies; (6) no chromosomal abnormality of couples; and (7) no contraindication for infliximab. In particular, these women had 3.2±2.0 IVF failures, had 1.3±1.6 abortions, and were of 36.0±3.5 years of age. Among the 11 women, 5 women had primary infertility and 6 women had a history of spontaneous abortions after IVF/ET.

Inflixmab (Remicade™) 3 mg/Kg was administered intravenously on cycle day one. Among the 11 women who underwent the infliximab therapy, 4 women failed to get pregnant and 7 became pregnant (63%). Among the 7 women who were pregnant, 1 woman delivered live born infant, 3 women currently have ongoing pregnancies, and 3 women aborted. 5 out of the 7 women who were pregnant conceived by IVF/ET, and the other 2 women conceived naturally. The average length to achieve a positive pregnancy from infliximab infusion was 21.1±10.7 weeks (from 6.1 weeks to 38 weeks).

The results demonstrate that serum TNF-α levels in these women decreased significantly after infliximab infusion (P<0.0001). Pre-infliximab infusion, the average serum TNF-α level was 332.8±173.2 pg/ml (from 85 to 663 pg/ml). In contrast, the average serum TNF-α level one week post-infliximab infusion was 15.7±4.8 pg/ml (from 7 to 25 pg/ml). Peripheral blood NK cytotoxicity, CD56+ NK cells and CD 19+/5+ B cells were not significantly different pre- and post-infliximab infusion.

Individual Case Studies

A female patient (Patient ID: OWH), age 35, was primary infertility with unknown etiology. Obstetrical history for the patient was: Gravida 0, para 0, Failed IVF 3. The patient was diagnosed with autoimmune thyroiditis with a history of rheumatoid arthritis, undifferentiated mixed connective tissue disorder, and methylene tetrahydrofolate reductase (MTHFR) gene homozygous mutation. The patient failed to become pregnant with lymphocyte immunotherapy (LIT), Enbrel™, prednisone, aspirin treatment in combination with IVF. The patient received a single Remicade™ intravenous infusion of 3 mg/Kg with the continuation of (LIT, aspirin, and Lovenox™. She got pregnant naturally in about 3 months after the therapy, but miscarried. About 8 months later, she received the Remicade™ treatment again in addition to aspirin, celexa, prednisone, folgard, metformin and thyroxine and got pregnant naturally again. A healthy boy 6 lb. 10 oz. was delivered by Cesarean section in 37 weeks.

### EXAMPLE 3

### Prophetic example of vaginal infliximab formulation

It is contemplated that the intravenous administration of infliximab can be substituted by vaginal administration of the infliximab using a formulation of the infliximab formulated in a gel or any other controlled release intrauterine vehicles or devices (e.g., foam or sponge).

### EXAMPLE 4

### Treatment of Infertility Using Etanercept

71 women with multiple implantation failures after IVF/ET cycles and elevated TNF-α level were selected for treatment. Group one, including 35 women, received prednisone, intravenous immunoglobulin G, and anticoagulants (heparin and/or aspirin). In addition to treatment with intravenous immunoglobulin G, and anticoagulants (heparin and/or aspirin), group two, including 36 women, received etanercept 25 mg subcutaneously, twice weekly at least four weeks prior to conception, instead of prednisone treatment.

Among the 71 women, the pregnancy rate and ongoing pregnancy rate of the immunotherapy group without etanercept treatment were 22/35 (63%) and 16/22 (73%), and those with etanercept treatment were 17/36 (47%) and 15/17 (82%), respectively. The pregnancy rates and ongoing pregnancy rates between these two groups were not significantly different (p=0.278, p=0.873, respectively). Both groups of women had significantly higher pregnancy rates compared to that of reported pregnancy rates of repeated IVF failures with further IVF attempt (12/70. 17.1%, p=0.000007) and zygote intrafallopian transfer (ZIFT) attempts (24/70, 34.2%, p=0.0199).

Individual Case Studies

A first female patient (Patient ID: TBO), age 32, had an obstetrical history at admission of 1 live birth and 4 miscarriages, 1 miscarriage due to ruptured ovarian cyst with a previous partner, 1 therapeutic abortion due to hepatitis A with a previous partner. Diagnosis included: relatively elevated Natural Killer cell cytotoxicity; thromboembolism in decidual vessel; presence of endometrial stromal hemorrhages; and undifferentiated mixed connective tissue disorder. The patient started Enbrel™ treatment at a dose of 25 mg subcutaneously twice weekly for 2 months (with preconception LIT, Lovenox™, baby aspirin, progesterone with post conception IVIg) and became pregnant after two months with natural cycle. A healthy girl 6 lb. 13 oz. was delivered at 38 weeks gestation with cesarean section.

A second female patient (Patient ID: CBE), age 31, had an obstetrical history at admission of gravida 0, and para 0. The patient had 6 IVF failures including: 4 failed IVFs; 1 failed IVF with heparin+baby aspirin only treatment (5^{th} IVF attempt); 1 failed IVF with lymphocyte immunization donor and paternal, aspirin+heparin treatment; and intravenous immunoglobulin G infusion treatment on cycle day 6 (6^{th} IVF attempt). The patient was diagnosed with inherited thrombophilia—Leiden Factor V homozygous mutation; HCG antibodies; and antiphospholipid antibody positivity At the 7th IVF attempt, the patient was on preconception Enbrel™ of 25 mg subcutaneously twice weekly in addition to donor and paternal lymphocyte immunotherapy, Lovenox™, baby aspirin, Prozac™, and human chorionic gonadotropin. A healthy girl 6 lb. 5 oz. was delivered at 37 weeks by vaginal delivery. Apgar score was 10/10.

### EXAMPLE 5

### Prophetic Example of Vaginal Etanercept Formulation

It is contemplated that the subcutaneous administration of etanercept can be substituted by vaginal administration of the etanercept using a formulation of the etanercept formulated in a gel or any other controlled release intrauterine vehicles or devices (e.g., foam or sponge).

### EXAMPLE 6

### Prophetic Examples of Using Other TNF-α Anatagonists for the Treatment of Infertility

It is contemplated that infliximab or etanercept in examples 1 to 5 can be substituted by any of the other TNF-α anatagonists disclosed in the present application, including D2E7, CDP571, structural analogs of thalidomide, and phosphodiesterase type IV inhibitors.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A method for treating infertility in a subject, the subject having a ratio of T helper 1 (Th1) immune response to T helper 2 (Th2) immune response, and the method comprises reducing the ratio of Th1 immune response to Th2 immune response in the subject to inhibit spontaneous abortion or implantation failure.

2. The method of claim 1, wherein the implantation failure occurs after assisted reproductive technology (ART) cycles.

3. The method of claim 2, wherein the ART is in vitro fertilization.

4. The method of claim 1, wherein the ratio of Th1 immune response to Th2 immune response in the subject is a ratio of absolute cell counts of a representative population of Th1 cells to a representative population of Th2 cells.

5. The method of claim 1, wherein the ratio of Th1 immune response to Th2 immune response in the subject is determined by measuring a ratio of the level of a Th1 cytokine to a Th2 cytokine.

6. The method of claim 5, wherein the levels of Th1 and Th2 cytokines are serum levels.

7. The method of claim 5, wherein the levels of Th1 and Th2 cytokines are intracellular levels.

8. The method of claim 1, wherein the method of reducing the ratio of Th1 immune response to Th2 immune response is to reduce the absolute counts of Th1 cells in the subject.

9. The method of claim 8, wherein the Th1 cell is a TNF-α expressing CD3+/CD4+ T-cell.

10. The method of claim 1, wherein the method of reducing the ratio of Th1 immune response to Th2 immune response is to increase the absolute counts of Th2 cells in the subject.

11. The method of claim 10, wherein the Th2 cell is an IL-4 expressing CD3+/CD8− T-cell.

12. The method of claim 1, wherein the method of reducing the ratio of Th1 immune response to Th2 immune response is to suppress the Th1 cytokines in the subject.

13. The method of claim 1, wherein the method of reducing the ratio of Th1 immune response to Th2 immune response is to enhance the level of Th2 cytokines in the subject.

14. The method of claim 12, wherein the Th1 cytokines are selected from the group consisting of IL-1, IL-2, TNF-α, and IFN-γ.

15. The method of claim 13, wherein the Th2 cytokines are selected from the group consisting of IL-4, IL-5, IL-6, IL-10.

16. The method of claim 5, wherein the Th1 to Th2 cytokine ratio is a ratio of IFN-γ to IL-4.

17. The method of claim 5, wherein the Th1 to Th2 cytokine ratio is a ratio of IFN-γ to IL-10.

18. The method of claim 5, wherein the Th1 to Th2 cytokine ratio is a ratio of TNF-α to IL-4.

19. The method of claim 5, wherein the Th1 to Th2 cytokine ratio is a ratio of TNF-α to IL-10.

20. The method of claim 5, wherein the method of reducing the count of Th1 cells is by administering an effective dose of an inhibitor of a costimulatory signal of a T-cell.

21. The method of claim 20, wherein the agent is an antibody to CD80.

22. The method of claim 20, wherein the agent is an antibody to CD86.

23. The method of claim 20, wherein the agent is an antibody to ICOS.

24. The method of claim 20, wherein the agent is a soluble form of CD28.

25. The method of claim 20, wherein the agent is a soluble form of ICOS.

26. The method of claim 20, wherein the agent is a soluble form of CTLA4.

27. The method of claim 10, wherein the method of increasing the count of Th2 cells is by administering an effective dose of a T helper 2-immuno-stimulatory nucleic acid.

28. The method of claim 12, wherein the method of suppressing the Th1 cytokines is by administering an effective dose of a Th1 cytokine antagonist to the subject.

29. The method of claim 28, wherein the Th1 cytokine antagonist is an inhibitor of the synthesis of the cytokine.

30. The method of claim 28, wherein the Th1 cytokine antagonist blocks the binding of the cytokine to its receptor.

31. The method of claim 28, wherein the Th1 cytokine antagonist inactivates the cytokine by binding to the cytokine.

32. The method of claim 28, wherein the Th1 cytokine antagonist is a polyclonal antibody.

33. The method of claim 28, wherein the Th1 cytokine antagonist is a monoclonal antibody.

34. The method of claim 28, wherein the Th1 cytokine antagonist is a soluble receptor of the cytokine.

35. The method of claim 28, wherein the Th1 cytokine antagonist is selected from the group consisting of: IL-1 antagonists, IL-2 antagonists, TNF-α antagonists, and IFN-γ antagonists.

36. The method of claim 35, wherein the TNF-α antagonist is infliximab.

37. The method of claim 35, wherein the TNF-α antagonist is etanercept.

38. The method of claim 35, wherein the TNF-α antagonist is D2E7.

39. The method of claim 35, wherein the TNF-α antagonist is CDP571.

40. The method of claim 35, wherein the TNF-α antagonist is CDP870.

41. The method of claim 35, wherein the TNF-α antagonist is a thalidomide analog.

42. The method of claim 35, wherein the TNF-α antagonist is a phosphodiesterase type IV inhibitor.

43. The method of claim 1, wherein the treatment of infertility further comprises enhancing embryo implantation, pregnancy, or birth rates of the subject.

44. The method of claim 1, wherein the treatment of infertility enhances the ability of the subject to carry at least one embryo to term.

45. The method of claim 1, wherein the subject is a human.

46. The method of claim 1, wherein the subject has had one or more previous spontaneous abortions or implantation failures.

47. The method of claim 46, wherein the implantation failures occur after ART cycles.

48. The method of claim 47, wherein the ART is in vitro fertilization.

49. The method of claim 1, wherein the subject undergoes natural conception.

50. The method of claim 1, wherein the subject undergoes ART cycles.

51. The method of claim 50, wherein the ART is in vitro fertilization.

52. The method of claim 1, wherein the subject undergoes ovulation induction cycles.

53. A method for treating infertility in a subject by inhibiting spontaneous abortion or implantation failure for enhancing embryo implantation, pregnancy, or birth rates, the method comprising administering a therapeutically effective dosage level of a TNF-α antagonist to the subject.

54. The method of claim 53, wherein the implantation failure occurs after ART cycles.

55. The method of claim 54, wherein the ART is in vitro fertilization.

56. The method of claim 53, further enhancing the ability of the subject to carry at least one embryo to term.

57. The method of claim 53, wherein the subject is a human.

58. The method of claim 53, wherein the subject has had one or more previous spontaneous abortions or implantation failures.

59. The method of claim 58, wherein the implantation failures occur after ART cycles.

60. The method of claim 59, wherein the ART is in vitro fertilization.

61. The method of claim 53, wherein the subject undergoes natural conception.

62. The method of claim 53, wherein the subject undergoes ART cycles.

63. The method of claim 62, wherein the ART is in vitro fertilization.

64. The method of claim 53, wherein the subject undergoes ovulation induction cycles.

65. The method of claim 53, wherein the TNF-α antagonist is infliximab.

66. The method of claim 53, wherein the TNF-α antagonist is etanercept.

67. The method of claim 53, wherein the TNF-α antagonist is D2E7.

68. The method of claim 53, wherein the TNF-α antagonist is CDP571.

69. The method of claim 53, wherein the TNF-α antagonist is CDP870.

70. The method of claim 53, wherein the TNF-α antagonist is a thalidomide analog.

71. The method of claim 53, wherein the TNF-α antagonist is a phosphodiesterase type IV inhibitor.

72. A pharmaceutical composition for treating infertility in a subject by inhibiting spontaneous abortion or implantation failure for enhancing embryo implantation, pregnancy, or birth rates, the composition comprising a TNF-α antagonist formulated in a formulation suitable for administration to the subject vaginally.

73. A method of enhancing the ability of a subject to carry at least one embryo to term comprising administering to the subject an effective dose of a TNF-α antagonist to inhibit TNF-A in the subject to inhibit spontaneous abortion or implantation failure.

74. The method of claim 73, wherein the implantation failure occurs after ART cycles.

75. The method of claim 74, wherein the ART is in vitro fertilization.

76. The method of claim 73, wherein the subject is a human.

77. The method of claim 73, wherein the subject has had one or more previous spontaneous abortions or implantation failures.

78. The method of claim 73, wherein the TNF-α antagonist is infliximab.

79. The method of claim 73, wherein the TNF-α antagonist is etanercept.

80. The method of claim 73, wherein the TNF-α antagonist is D2E7.

81. The method of claim 73, wherein the TNF-α antagonist is CDP571.

82. The method of claim 37, wherein the TNF-α antagonist is CDP870.

83. The method of claim 73, wherein the TNF-α antagonist is a thalidomide analog.

84. The method of claim 73, wherein the TNF-α antagonist is a phosphodiesterase type IV inhibitor.

85. A pharmaceutical composition for enhancing the ability of a subject to carry at least one embryo to term comprising a TNF-α antagonist to inhibit TNF-α in the subject to inhibit spontaneous abortion or implantation failure, wherein the TNF-α is formulated in formulation suitable for administration to the subject vaginally.

86. A method for treating infertility in a subject by inhibiting spontaneous abortion or implantation failure for enhancing embryo implantation, pregnancy, or birth rates, the method comprising administering a therapeutically effective dosage level of infliximab to the subject.

87. The method of claim 86, wherein the implantation failure occurs after ART cycles.

88. The method of claim 86, wherein the ART is in vitro fertilization.

89. The method of claim 86, further enhancing the ability of the subject to carry at least one embryo to term.

90. The method of claim 86, wherein the subject is a human.

91. The method of claim 86, wherein the subject has had one or more previous spontaneous abortions or implantation failures.

92. The method of claim 91, wherein the implantation failures occur after ART cycles.

93. The method of claim 92, wherein the ART is in vitro fertilization.

94. The method of claim 86, wherein the subject undergoes natural conception.

95. The method of claim 86, wherein the subject undergoes ART cycles.

96. The method of claim 95, wherein the ART is in vitro fertilization.

97. The method of 86, wherein the subject undergoes ovulation induction cycles.

98. The method of claim 86, wherein the therapeutically effective dosage level of infliximab is from about 3 mg/Kg to about 10 mg/Kg.

99. The method of claim 86, wherein the administration of infliximab is performed by delivering a therapeutically effective dosage level of infliximab intravenously.

100. The method of claim 86, wherein the administration of infliximab is performed by delivering a therapeutically effective dosage level of infliximab subcutaneously.

101. The method of claim 86, wherein the administration of infliximab is performed by delivering a therapeutically effective dosage level of infliximab vaginally.

102. The method of claim 101, wherein the infliximab is in a gel form.

103. The method of claim 86, wherein the administration of infliximab is performed by delivering a therapeutically effective dosage of infliximab at least once prior to index conception cycle day one.

104. The method of claim 86, wherein the step of the administration of infliximab is performed by delivering a therapeutically effective dosage of infliximab at least once on index conception cycle day one.

105. The method of claim 86, wherein the administration of infliximab is performed by delivering a therapeutically effective dosage of infliximab at least once after index conception cycle day one.

106. The method of claim 86, wherein the subject further receives lymphocyte immunization or autoimmune treatment.

107. The method of claim 86, wherein the subject further receives intravenous immunoglobulin G.

108. The method of claim 86, wherein the subject further receives at least one anticoagulant.

109. The method of claim 108, wherein one of the anticoagulants is heparin.

110. The method of claim 108, wherein one of the anticoagulants is aspirin.

111. The method of claim 86, wherein the subject further receives prednisone.

112. A pharmaceutical composition for treating infertility in a subject by inhibiting spontaneous abortion or implantation failure for enhancing embryo implantation, pregnancy, or birth rates, the composition comprising infliximab formulated in a formulation suitable for administration to the subject vaginally.

113. A method for treating infertility in a subject by inhibiting spontaneous abortion or implantation failure for enhancing embryo implantation, pregnancy, or birth rates, the method comprising administering a therapeutically effective dosage level of etanercept to the subject.

114. The method of claim 113, wherein the implantation failure occurs after ART cycles.

115. The method of claim 114, wherein the ART is in vitro fertilization

116. The method of claim 113, further enhancing the ability of the subject to carry at least one embryo to term.

117. The method of claim 113, wherein the subject is a human.

118. The method of claim 113, wherein the subject has had one or more previous spontaneous abortions or implantation failures.

119. The method of claim 118, wherein the implantation failures occur after ART cycles.

120. The method of claim 119, wherein the ART is in vitro fertilization.

121. The method of claim 113, wherein the subject undergoes natural conception.

122. The method of claim 113, wherein the subject undergoes ART cycles.

123. The method of claim 122, wherein the ART is in vitro fertilization.

124. The method of claim 113, wherein the subject undergoes ovulation induction cycles.

125. The method of claim 113, wherein the therapeutically effective dosage level of etanercept is from about 3 mg to about 50 mg.

126. The method of claim 113, where the administration of etanercept is performed by delivering a therapeutically effective dosage level of etanercept subcutaneously.

127. The method of claim 113, wherein the administration of etanercept is performed by delivering a therapeutically effective dosage level of etanercept vaginally.

128. The method of claim 127, wherein the etanercept is in a gel form.

129. The method of claim 113, wherein the administration of etanercept is performed by delivering a therapeutically effective dosage of etanercept at least once prior to index conception cycle day one.

130. The method of claim 113, wherein the administration of etanercept is performed by delivering a therapeutically effective dosage of etanercept at least once on index conception cycle day one.

131. The method of claim 113, wherein the administration of etanercept is performed by delivering a therapeutically effective dosage of etanercept at least once after index conception cycle day one.

132. The method of claim 113, wherein the subject further receives lymphocyte immunization or autoimmune treatment.

133. The method of claim 113, wherein the subject further receives intravenous immunoglobulin G.

134. The method of claim 113, wherein the subject further receives at least one anticoagulant.

135. The method of claim 134, wherein one of the anticoagulants is heparin.

136. The method of claim 134, wherein one of the anticoagulants is aspirin.

137. The method of claim 113, wherein the subject further receives prednisone.

138. A pharmaceutical composition for treating infertility in a subject by inhibiting spontaneous abortion or implantation failure for enhancing embryo implantation, pregnancy, or birth rates, the composition comprising etanercept formulated in a formulation suitable for administration to the subject vaginally.

139. A method for treating infertility in a subject by inhibiting spontaneous abortion or implantation failure for enhancing embryo implantation, pregnancy, or birth rates, the method comprising administering a therapeutically effective dosage level of D2E7 to the subject.

140. The method of claim 139, wherein the implantation failure occurs after ART cycles.

141. The method of claim 140, wherein the ART is in vitro fertilization.

142. The method of claim 139, further enhancing the ability of the subject to carry at least one embryo to term.

143. The method of claim 139, wherein the subject is a human.

144. The method of claim 139, wherein the subject has had one or more previous spontaneous abortions or implantation failures.

145. The method of claim 144, wherein the implantation failures occur after ART cycles.

146. The method of claim 145, wherein the ART is in vitro fertilization.

147. The method of claim 139, wherein the subject undergoes natural conception.

148. The method of claim 139, wherein the subject undergoes ART cycles.

149. The method of claim 148, wherein the ART is in vitro fertilization.

150. The method of claim 139, wherein the subject undergoes ovulation induction cycles.

151. The method of claim 124, wherein the dosage level of D2E7 is from about 5 mg to about 50 mg.

152. The method of claim 139, wherein the administration of D2E7 is performed by delivering a therapeutically effective dosage level of D2E7 subcutaneously.

153. The method of claim 139, wherein the administration of D2E7 is performed by delivering a therapeutically effective dosage level of D2E7 intravenously.

154. The method of claim 139, wherein the administration of D2E7 is performed by delivering a therapeutically effective dosage level of D2E7 vaginally.

155. The method of claim 139, wherein the administration of D2E7 is performed by delivering a therapeutically effective dosage of D2E7 at least once prior to index conception cycle day one.

156. The method of claim 139, wherein the administration of D2E7 is performed by delivering a therapeutically effective dosage of D2E7 at least once on index conception cycle day one.

157. The method of claim 139, wherein the administration of D2E7 is performed by delivering a therapeutically effective dosage of D2E7 at least once after index conception cycle day one.

158. The method of claim 139, wherein the subject further receives lymphocyte immunization or autoimmune treatment.

159. The method of claim 139, wherein the subject further receives intravenous immunoglobulin G.

160. The method of claim 139, wherein the subject further receives at least one anticoagulant.

161. The method of claim 160, wherein one of the anticoagulants is heparin.

162. The method of claim 160, wherein one of the anticoagulants is aspirin.

163. The method of claim 139, wherein the subject further receives prednisone.

164. A pharmaceutical composition for treating infertility in a subject by inhibiting spontaneous abortion or implantation failure for enhancing embryo implantation, pregnancy, or birth rates, the composition comprising D2E7 formulated in al formulation suitable for administration to the subject vaginally.

165. A method for treating infertility in a subject to by inhibiting spontaneous abortion or implantation failure for enhancing embryo implantation, pregnancy, or birth rates, the method comprising administering a therapeutically effective dosage level of CDP571 to the subject.

166. The method of claim 165, wherein the implantation failure occurs after ART cycles.

167. The method of claim 166, wherein the ART is in vitro fertilization.

168. The method of claim 165, further enhancing the ability of the subject to carry at least one embryo to term.

169. The method of claim 165, wherein the subject is a human.

170. The method of claim 165, wherein the subject has had one or more previous spontaneous abortions, or implantation failures.

171. The method of claim 170, wherein the implantation failures occur after ART cycles.

172. The method of claim 171, wherein the ART is in vitro fertilization.

173. The method of claim 170, wherein the subject undergoes natural conception.

174. The method of claim 165, wherein the administration of CDP571 is performed by delivering a therapeutically effective dosage level of CDP571 subcutaneously.

175. The method of claim 165, wherein the administration of CDP571 is performed by delivering a therapeutically effective dosage level of CDP571 vaginally.

176. A pharmaceutical composition for treating infertility in a subject by inhibiting spontaneous abortion or implantation failure for enhancing embryo implantation, pregnancy, or birth rates, the composition comprising CDP571 formulated in a formulation suitable for administration to the subject vaginally.

177. A method for treating infertility in a subject by inhibiting spontaneous abortion or implantation failure for enhancing embryo implantation, pregnancy, or birth rates, the method comprising administering a therapeutically effective dosage level of CDP870 to the subject.

178. The method of claim 177, wherein the implantation failure occurs after ART cycles.

179. The method of claim 178, wherein the ART is in vitro fertilization.

180. The method of claim 177, further enhancing the ability of the subject to carry at least one embryo to term.

181. The method of claim 177, wherein the subject is a human.

182. The method of claim 177, wherein the subject has had one or more previous spontaneous abortions or implantation failures.

183. The method of claim 177, wherein the subject undergoes natural conception.

184. The method of claim 182, wherein the implantation failures occur after ART cycles.

185. The method of claim 184, wherein the ART is in vitro fertilization.

186. The method of claim 177, wherein the administration of CDP870 is performed by delivering a therapeutically effective dosage level of CDP870 subcutaneously.

187. The method of claim 177, wherein the administration of CDP870 is performed by delivering a therapeutically effective dosage level of CDP870 vaginally.

188. A pharmaceutical composition for treating infertility in a subject by inhibiting spontaneous abortion or implantation failure for enhancing embryo implantation, pregnancy, or birth rates, the composition comprising CDP870 formulated in a formulation suitable for administration to the subject vaginally.

189. A method for treating infertility in a subject by inhibiting spontaneous abortion or implantation failure for enhancing embryo implantation, pregnancy, or birth rates, the method comprising administering a therapeutically effective dosage level of a thalidomide analog to the subject.

190. The method of claim 189, wherein the implantation failure occurs after ART cycles.

191. The method of claim 190, where in the ART is in vitro fertilization

192. The method of claim 189, further enhancing the ability of the subject to carry at least one embryo to term.

193. The method of claim 189, wherein the subject is a human.

194. The method of claim 189, wherein the subject undergoes natural conception.

195. The method of claim 189, wherein the subject has had one or more previous spontaneous abortions or implantation failures.

196. The method of claim 194, wherein the implantation failures occur after ART cycles.

197. The method of claim 196, wherein the ART is in vitro fertilization.

198. The method of claim 189, wherein the administration of the thalidomide analog is performed by delivering a therapeutically effective dosage level of the thalidomide analog orally, vaginally, subcutaneously or intravenously.

199. The method of claim 189, wherein the administration of the thalidomide analog is performed subcutaneously in the woman wherein the dosage level is from about 50 mg/Kg to about 800 mg/Kg.

200. The method of claim 189, wherein the therapeutically effective dosage level is sufficient to produce a blood level of the thalidomide analog of at least 0.1 μg/ml.

201. A pharmaceutical composition for treating infertility in a subject by inhibiting spontaneous abortion or implantation failures for enhancing embryo implantation, pregnancy, or birth rates, the composition comprising a thalidomide analog formulated in a formulation suitable for administration to the subject vaginally.

202. A method for treating infertility in a subject by inhibiting spontaneous abortion or implantation failure for enhancing embryo implantation, pregnancy, or birth rates, the method comprising administering a therapeutically effective dosage level of a phosphodiesterase type IV inhibitor to the subject.

203. The method of claim 202, wherein the implantation failure occurs after ART cycles.

204. The method of claim 203, wherein the ART is in vitro fertilization cycles.

205. The method of claim 202, further enhancing the ability of the subject to carry at least one embryo to term.

206. The method of claim 202, wherein the subject undergoes natural conception.

207. The method of claim 202, wherein the subject is a human.

208. The method of claim 202, wherein the subject has had one or more previous spontaneous abortions or implantation failures.

209. The method of claim 208, wherein implantation failures occur after ART cycles.

210. The method of claim 209, wherein the ART is in vitro fertilization.

211. A pharmaceutical composition for treating infertility in a subject by inhibiting spontaneous abortion or implantation failure for enhancing embryo implantation, pregnancy, or birth rates, the composition comprising a phosphodiesterase type IV inhibitor formulated in a formulation suitable for administration to the subject vaginally.

212. A method for diagnosing infertility in a subject with recurrent spontaneous abortions or one or more implantation failures comprising determining a ratio of Th1 and Th2 immune responses of the subject and comparing the ratio with that from subjects with normal pregnancies to determine if the subject is at risk of infertility or if the subject is suitable for treatment of the infertility by reducing ratio of Th1 to Th2 immune responses in the subject.

213. The method of claim 212, wherein the implantation failures occur after ART cycles.

214. The method of claim 213, wherein the ART is in vitro fertilization.

215. The method of claim 212, wherein the Th1 immune response is measured by the absolute cell counts of Th1 cytokine expressing T-cells, and the Th2 immune response is measured by the absolute cell counts of Th2 cytokine expressing T-cells.

216. The method of claim 215, wherein the Th1 and Th2 immune responses are measured by flow cytometry analysis.

217. The method of claim 215, wherein the Th1 cytokine expressing T-cell is a TNF-α expressing CD3+/CD4+ T-cell.

218. The method of claim 215, wherein the Th2 cytokine expressing T-cell is a IL-4 expressing CD3+/CD4+ T-cell.

219. The method of claim 212, wherein the ratio of Th1 immune response to Th2 immune response is a ratio of the levels of Th1 and Th2 cytokines.

220. The method of claim 219, wherein the levels of Th1 and Th2 cytokines are serum levels.

221. The method of claim 219, wherein the levels of Th1 and Th2 cytokines are intracellular levels.

222. The method of claim 219, wherein the Th1 cytokine is IL-1.

223. The method of claim 219, wherein the Th1 cytokine is IL-2.

224. The method of claim 219, wherein the Th1 cytokine is TNF-α.

225. The method of claim 219, wherein the Th1 cytokine is IFN-y.

226. The method of claim 219, wherein the Th2 cytokine is IL-4.

227. The method of claim 219, wherein the Th2 cytokine is IL-5.

228. The method of claim 219, wherein the Th2 cytokine is IL-6.

229. The method of claim 219, wherein the Th2 cytokine is IL-10.

230. The method of claim 219, wherein the Th1 to Th2 cytokine ratio is the ratio of IFN-y to IL-4.

231. The method of claim 219, wherein the Th1 to Th2 cytokine ratio is the ratio of IFN-γ to IL-10.

232. The method of claim 219, wherein the Th1 to Th2 cytokine ratio is the ratio of TNF-α to IL-4.

233. The method of claim 219, wherein the Th1 to Th2 cytokine ratio is the ratio of TNF-α to IL-10.

234. A diagnostic kit for diagnosing infertility with recurrent spontaneous abortions or one or more implantation failure in a subject, the kit comprising:
(a) means for determining Th1 immune response; and
(b) means for determining Th2 immune response.

235. The diagnostic kit of claim 234, wherein the implantation failures occur after ART cycles.

236. The diagnostic kit of claim 235, wherein the ART is in vitro fertilization.

237. The diagnostic kit of claim 234, further providing a ratio of Th1 to Th2 immune responses in a population of other subjects with normal pregnancies.

238. The diagnostic kit of claim 234, wherein the Th1 immune response is the levels of Th1 cytokines in the subject, the means for determining the Th1 immune response comprises a Th1 cytokine antibody, the Th2 immune response is the levels of Th2 cytokines in the subject, and the means for determining the Th2 immune response comprises a Th2 cytokine antibody.

239. The diagnostic kit of claim 234, wherein the antibody is a polyclonal or monoclonal antibody or a fragment thereof.

240. A method for determining whether a treatment of infertility in a subject with recurrent spontaneous abortions or one or more implantation failures by reducing the ratio of Th1 to Th2 immune responses is having the desired effect of enhancing embryo implantation, pregnancy, or birth rates in the subject, the method comprising the steps of:
(a) determining the ratio of the level of Th1 immune response to the level of Th2 immune response of the subject before the treatment;
(b) determining the ratio of the level of Th1 immune response to the level of Th2 immune response of the subject after the treatment;
(c) determining whether the ratio of Th1 to Th2 immune responses in the subject after the treatment is lower than that in the subject before the treatment to determine if the treatment has the desired effect.

241. The method of claim 240, wherein the implantation failures occur after ART cycles.

242. The method of claim 241, wherein the ART is in vitro fertilization.

243. The method of claim 240, wherein the Th1 immune response is measured by the absolute cell counts of Th1 cytokine expressing T-cells and the Th2 immune response is measured by the absolute cell counts of Th2 cytokine expressing T-cells.

244. The method of claim 243, wherein the Th1 and Th2 immune responses are measured by flow cytometry analysis.

245. The method of claim 243, wherein the Th1 cytokine expressing T-cell is a TNF-α expressing CD3+/CD4+ T-cell.

246. The method of claim 243, wherein the Th2 cytokine expressing T-cell is a IL-4 expressing CD3+/CD4+ T-cell.

247. The method of claim 243, wherein the ratio of Th1 immune response to Th2 immune response is a ratio of the level of Th1 and Th2 cytokines.

248. The method of claim 247, wherein the levels of Th1 and Th2 cytokines are serum levels.

249. The method of claim 247, wherein the levels of Th1 and Th2 cytokines are intracellular levels.

250. The method of claim 247, wherein the Th1 cytokine is IL-1.

251. The method of claim 247, wherein the Th1 cytokine is IL-2.

252. The method of claim 247, wherein the Th1 cytokine is TNF-α.

253. The method of claim 247, wherein the Th1 cytokine is IFN-γ.

254. The method of claim 247, wherein the Th2 cytokine is IL-4.

255. The method of claim 247, wherein the Th2 cytokine is IL-5.

256. The method of claim 247, wherein the Th2 cytokine is IL-6.

257. The method of claim 247, wherein the Th2 cytokine is IL-10.

258. A diagnostic method for determining whether a TNF-α antagonist therapy will likely enhance embryo implantation, pregnancy, or birth rates in a subject with recurrent spontaneous abortions or one or more implantation failures, the method comprising:
(a) measuring a level of TNF-α in the subject;
(b) determining whether the level of TNF-α in the subject is higher than that in other subjects with normal pregnancies.

259. The method of claim 258, wherein the implantation failures occur after ART cycles.

260. The method of claim 259, wherein the ART is in vitro fertilization.

261. The method of claim 258, wherein the level of TNF-α is determined by a method using a TNF-α antibody.

262. The method of claim 261, wherein the antibody is a polyclonal or monoclonal antibody or a fragment thereof.

263. The method of claim 258, wherein the level of TNF-α is serum level.

264. The method of claim 263, wherein the serum level of TNF-α of the subject is considered higher than the level in other subjects with normal pregnancies when the level in the subject is higher than 12 pg/ml.

265. The method of claim 258, wherein the level of TNF-α is intracellular level.

266. A diagnostic kit of claim 258, comprising a means for measuring the level of TNF-α.

267. The diagnostic kit of claim 266, wherein the means for measuring the level of TNF-α is an antibody.

268. The diagnostic kit of claim 267, wherein the antibody is a polyclonal or a monoclonal antibody or a fragment thereof.

269. A method for determining whether a TNF-α antagonist treatment of infertility in a subject with recurring spontaneous abortions or one or more implantatioin failures is having the desired effect of enhancing embryo implantation, pregnancy, or birth rates in the subject, the method comprising the steps of:
(a) measuring the level of TNF-α in the subject before the TNF-α antagonist treatment;
(b) measuring the level of TNF-α in the subject after the TNF-α antagonist treatment;
(c) determining whether the level of TNF-α in the subject after the TNF-α antagonist treatment is lower than that in the subject before the treatment.

270. The method of claim 269, wherein the implantation failures occur after ART cycles.

271. The method of claim 270, wherein the ART is in vitro fertilization.

272. The method of claim 269, wherein the level of TNF-α is serum level.

273. The method of claim 269, wherein the level of TNF-α is intracellular level.

274. A diagnostic kit of claim 269, comprising a means for measuring the level of TNF-α.

275. The diagnostic kit of claim 274, wherein the means for measuring the level of TNF-α is an antibody.

276. The diagnostic kit of claim 275, wherein the antibody is a polyclonal or monoclonal antibody or a fragment thereof.
